(19) United States
(12) Patent Application Publication
(10) Pub. No.: US 2023/0265066 A1
(43) Pub. Date: Aug. 24, 2023

(54) NAPHTHOPYRAN-CONTAINING COMPOUND, POLYMER, MIXTURE, COMPOSITION AND USE THEREOF IN WATER-SOLUBLE PHOTOCHROMIC MATERIALS

(71) Applicant: Nano and Advanced Materials Institute Limited, Hong Kong (HK)

(72) Inventors: Chi Ming LEUNG, Hong Kong (HK); Chun Hei WONG, Hong Kong (HK); Chi Ho KWOK, Hong Kong (HK); Chenmin LIU, Hong Kong (HK)

(21) Appl. No.: 18/180,149

(22) Filed: Mar. 8, 2023

Related U.S. Application Data
(63) Continuation-in-part of application No. 17/992,924, filed on Nov. 22, 2022.
(60) Provisional application No. 63/318,290, filed on Mar. 9, 2022.
(60) Provisional application No. 63/358,234, filed on Jul. 5, 2022.
(60) Provisional application No. 63/282,726, filed on Nov. 24, 2021.
(60) Provisional application No. 63/358,234, filed on Jul. 5, 2022.

(51) Int. Cl. C07D 311/92; C09K 9/02; C07D 311/94; C07D 491/052

(52) U.S. Cl. CPC C07D 311/92; C09K 9/02; C07D 311/94; C07D 491/052; C09K 2211/1018

(57) ABSTRACT

Provided herein are a series of water-soluble photochromic naphthopyran compounds, a mixture and a polymer thin film comprising the same, and applications thereof in photochromic materials, especially water-soluble photochromic material. The present photochromic naphthopyran compounds display excellent water solubility in neutral pH environment, rapid light response and fast thermal bleaching.

### CROSS-REFERENCE TO RELATED APPLICATIONS

The present application is a continuation-in-part of U.S. Non-Provisional patent application Ser. No. 17/992,924 filed 22 Nov. 2022, which claims priority from U.S. provisional patent application Ser. No. 63/358,234 filed 5 Jul. 2022 and U.S. provisional patent application Ser. 63/282,726 filed 24 Nov. 2021, and claims priority from a U.S. provisional patent application Ser. No. 63/318,290 filed 9 Mar. 2022, and the disclosures of which are incorporated by reference in their entirety.

### TECHNICAL FIELD

The present invention relates to photochromic organic compounds, a mixture, and a polymer thin film comprising the same, and uses and applications thereof in water-soluble photochromic materials. More specifically, the present photochromic organic compounds contain water-soluble naphthopyrans.

### BACKGROUND OF THE INVENTION

Photochromism is the light-induced, reversible transformation of a compound/molecule between two forms, A and B, with different absorption spectra, activated by absorption of radiation of a certain range of wavelength.

Photochromic compounds, such as spiropyran, spirooxazine and naphthopyran, can be activated by UV light source, either UVA or UVB, to display color changes. The wavelength of such light sources is typically about 320 nm to 390 nm. Upon absorption of UV light, the structure of photochromic compound changes and switches from the colorless ring-closed form to the colored ring-opened form. Currently, the most widely used commercial areas are photoelectrical information storage unit, light-induced molecular switch, intelligent window film coating, color-changing lens for glasses, color-changing clothing, high-end cosmetics, photochromic ink paint, imaging equipment and anti-counterfeiting ink.

Photochromic materials can be divided into T-type and P-type, where the T-type material is thermodynamically unstable and will undergo reverse reaction and return back to the original state upon removal of the source of irradiation; while P-type material will undergo reversible reaction only by the irradiation of light source with another wavelength. Naphthopyrans as T-type photochromic compounds have a lot of advantages, such as ease to synthesize, rapid light response as well as high reliability, and have thus been used in the fields of photochromic lenses, color-changing clothing and handicrafts.

It is observed that most of the commercially available photochromic product require organic solvents, most of which are hazardous substances that require special treatment and disposal procedures. While water-soluble photochromic materials can be used as an alternative choice for biological or environmentally friendly applications and can be widely used in cosmetics industry and textile and clothing industry, the development of water-soluble naphthopyran compounds is very limited, due to their rigid conjugated groups such as benzene ring and naphthalene ring, thus lacking flexibility and water solubilizing group and hence their extremely low water solubility. While water-soluble naphthopyrans and benzopyrans with the incorporation of quaternary alkylamino salts have been disclosed, an acidic condition is required in order to render water solubility.

In view of the disadvantages of the existing photochromic compounds in photochromic articles, there is a need for developing photochromic naphthopyran compounds with good water solubility in neutral pH environment, rapid light response and fast thermal bleaching. Such new photochromic compounds could be used in printing inks for security and anti-counterfeit measures as well as lenses, toys, and decorative articles. The present invention addresses this need.

### SUMMARY OF THE INVENTION

Provided herein are photochromic organic compounds, a mixture, and a polymer thin film comprising the same, and uses and applications thereof in water-soluble photochromic materials. In a first aspect, the present invention provides a water-soluble photochromic naphthopyran compound having a general structural formula represented by Formula (1):

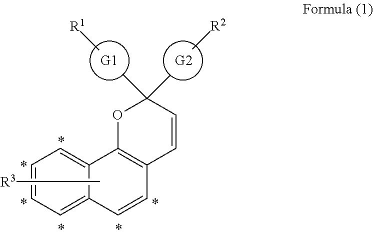

wherein G1 is selected from the group consisting of an aromatic containing 5 to 20 ring atoms, a heteroaromatic containing 5 to 20 ring atoms, or a non-aromatic ring system containing 5 to 20 ring atoms; the G1 has a substituent R¹, and the R¹ is the same or different in multiple occurrences;
wherein G2 is selected from the group consisting of an aromatic containing 5 to 20 ring atoms, a heteroaromatic containing 5 to 20 ring atoms, or a non-aromatic ring system containing 5 to 20 ring atoms; the G2 has a substituent R², and the R² is the same or different in multiple occurrences;
wherein “*” represent possible bonding locations of Formula (1) with following chemical formula:

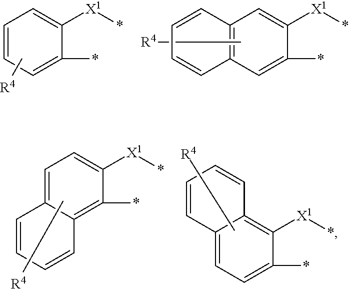

wherein X¹ is selected from CR⁵CR⁶, O, S, NR⁷, Se, S(═O)₂, C═O, S═O, PR^{8.},
wherein R¹, R², R³, R⁴, R⁵, R⁶, R⁷, R⁸ are independently selected from the group consisting of hydrogen, deuterium, a halogen atom, water-solubilizing group, —OCH₃, —NO₂, —C≡N, —N≡C, a linear alkyl group containing 1 to 20 carbon atoms, a branched alkyl group containing 1 to 20 carbon atoms, and a linear alkenyl group containing 1 to 20 carbon atoms, a branched alkenyl group containing 1 to 20 carbon atoms, an alkane ether group containing 1 to 20 carbon atoms, an aromatic containing 1 to 20 carbon atoms, a heteroaromatic containing 1 to 20 carbon atoms or a non-aromatic ring system containing 1 to 20 carbon atoms; and
wherein R¹, R², R³, R⁴, R⁵, R⁶, R⁷, R⁸ independently contain at least one water-solubilizing group selected from —SO₃H, —COOH, —OH, —NH₂, —NCₙH₂ₙ₊₁H⁺, —N(CₙH₂ₙ₊₁)₃⁺, —SO₃Na, —OSO₃H , —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin.

In one embodiment, G1 and G2 in Formula (1) of said photochromic napthopyran compound is selected from the following formulae:

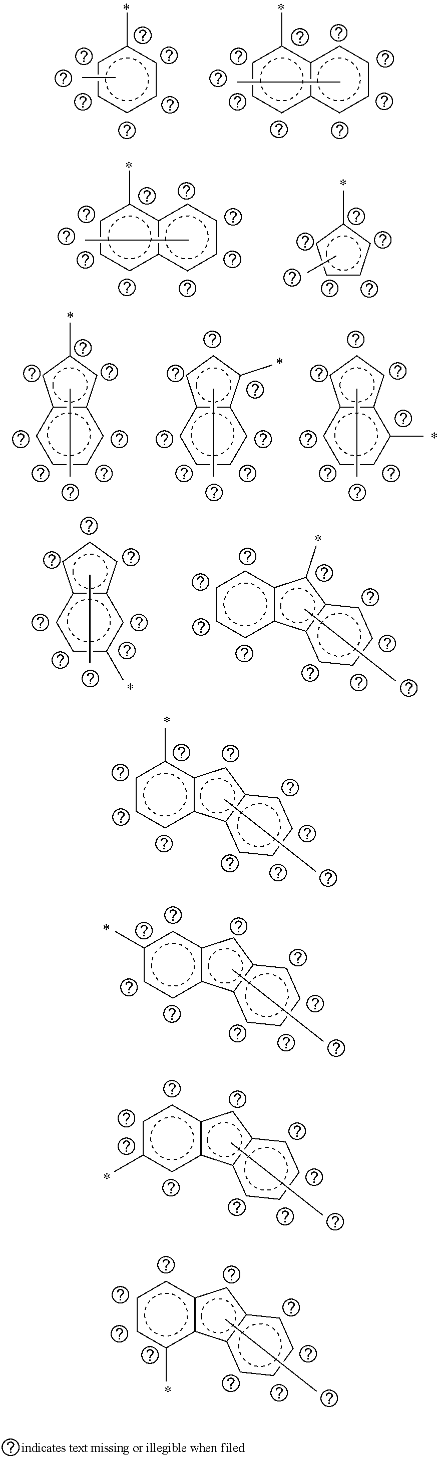

wherein # represents the bonding location in Formula (1);
wherein Z¹-Z⁹ are independently or jointly selected from the group consisting of carbon, nitrogen, oxygen, silicon, boron, sulfur or phosphorus atom;
wherein R⁹ are independently or jointly selected from the group consisting of hydrogen, deuterium, a halogen atom, —OCH₃, —NO₂, —C≡N, —N≡C, —NPh₂, N-carbazole, dibenzofuran, dibenzothiophene, —SO₃H, —OH, —NCₙH₂ₙ₊₁H⁺, —N(CₙH₂ₙ₊₁)₃⁺, —SO₃Na, —OSO₃H , —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin or a linear alkyl group containing 1 to 20 carbon atoms, a branched alkyl group containing 1 to 20 carbon atoms, and a linear alkenyl group containing 1 to 20 carbon atoms, a branched alkenyl group containing 1 to 20 carbon atoms, an alkane ether group containing 1 to 20 carbon atoms, an aromatic containing 1 to 20 carbon atoms, a heteroaromatic containing 1 to 20 carbon atoms or a non-aromatic ring system containing 1 to 20 carbon atoms.

In other embodiment, the photochromic naphthopyran compound contains a structure represented by one of general formulae (A-1) to (A-15):

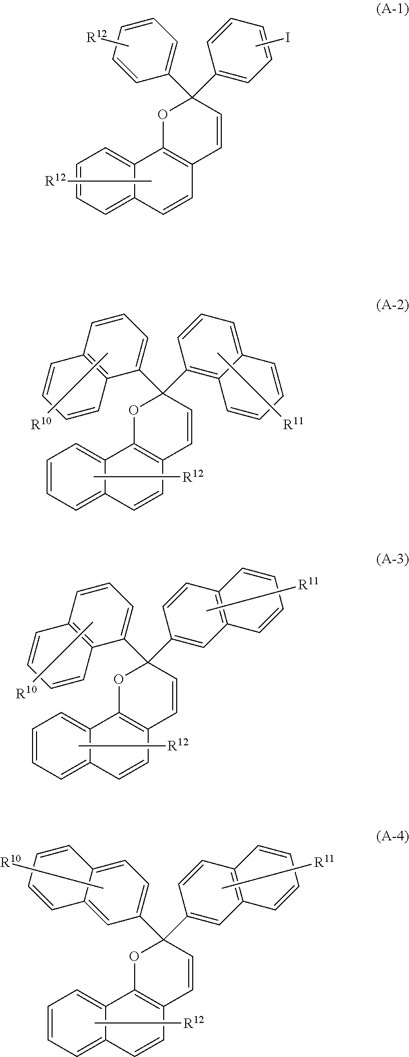

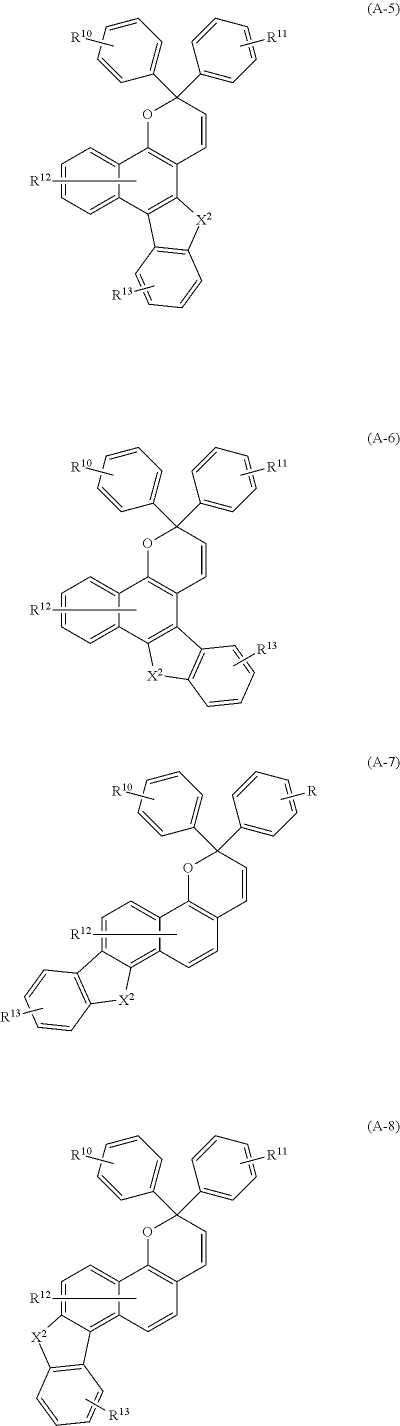

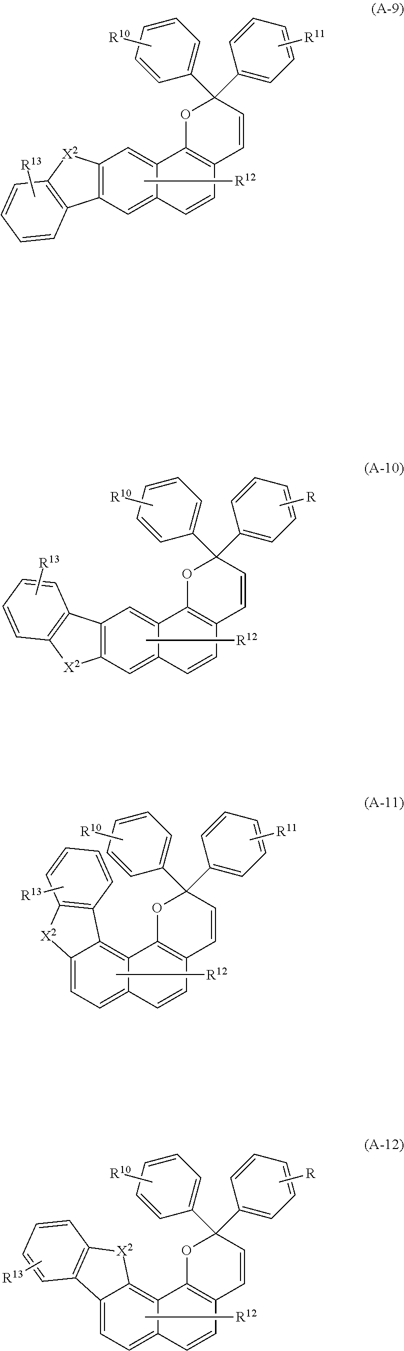

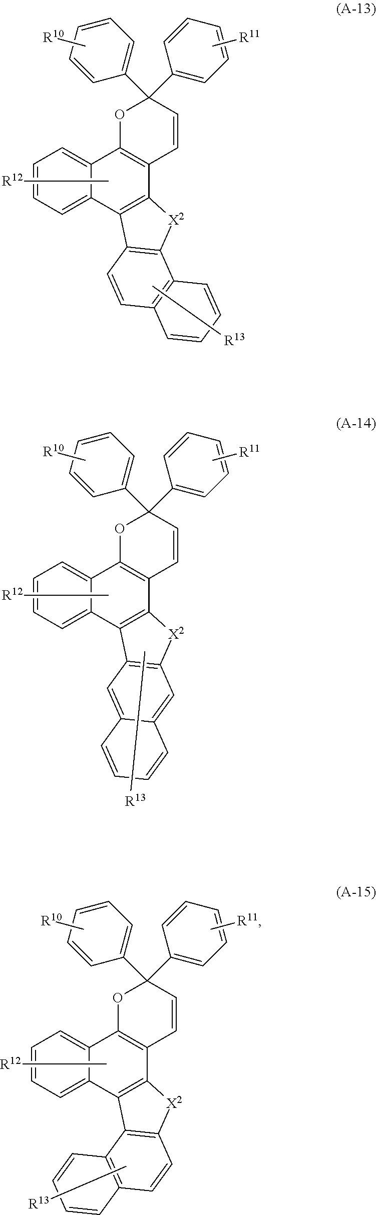

wherein R¹⁰, R¹¹, R¹², R¹³ are independently or jointly selected from the group consisting of hydrogen, deuterium, a halogen atom, —OCH₃, —NO₂, —C≡N, —N≡C, —NPh₂, N-carbazole, dibenzofuran, dibenzothiophene, —SO₃H, —OH, —NCₙH₂ₙ₊₁H⁺, —N(CₙH₂ₙ₊₁)₃⁺, —SO₃Na, —OSO₃H , —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin or a linear alkyl group containing 1 to 20 carbon atoms, a branched alkyl group containing 1 to 20 carbon atoms, and a linear alkenyl group containing 1 to 20 carbon atoms, a branched alkenyl group containing 1 to 20 carbon atoms, an alkane ether group containing 1 to 20 carbon atoms, an aromatic containing 1 to 20 carbon atoms, a heteroaromatic containing 1 to 20 carbon atoms or a non-aromatic ring system containing 1 to 20 carbon atoms;
wherein X² is selected from CR⁵CR⁶, O, S, NR⁷, Se, S(═O)₂, C═O, S═O, PR⁸.

In a further embodiment, at least one R¹⁰ or R¹² or R¹² or R¹³ is selected from —SO₃H, —OH, —NCₙH₂ₙ₊₁H⁺, —N(CₙH₂ₙ₊₁)₃⁺, —SO₃Na, —OSO₃H, —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin.

In yet another embodiment, the photochromic naphthopyran compound is selected from the following chemical structures:

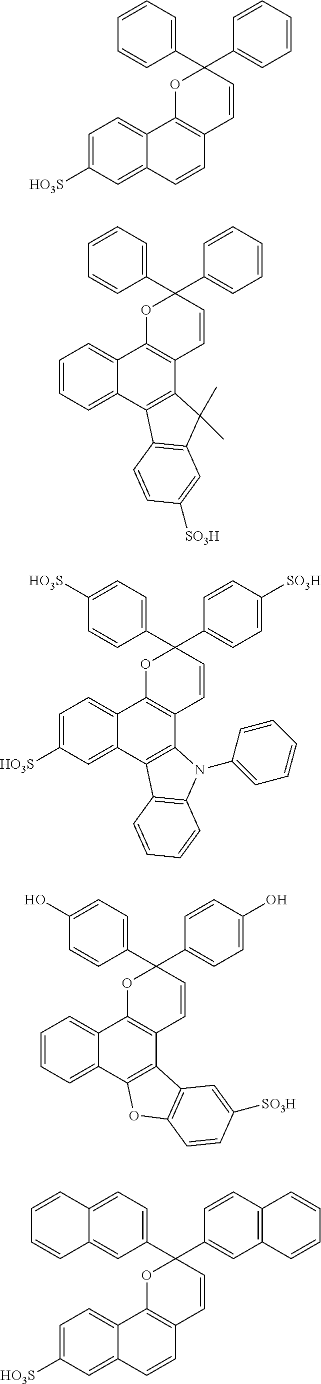

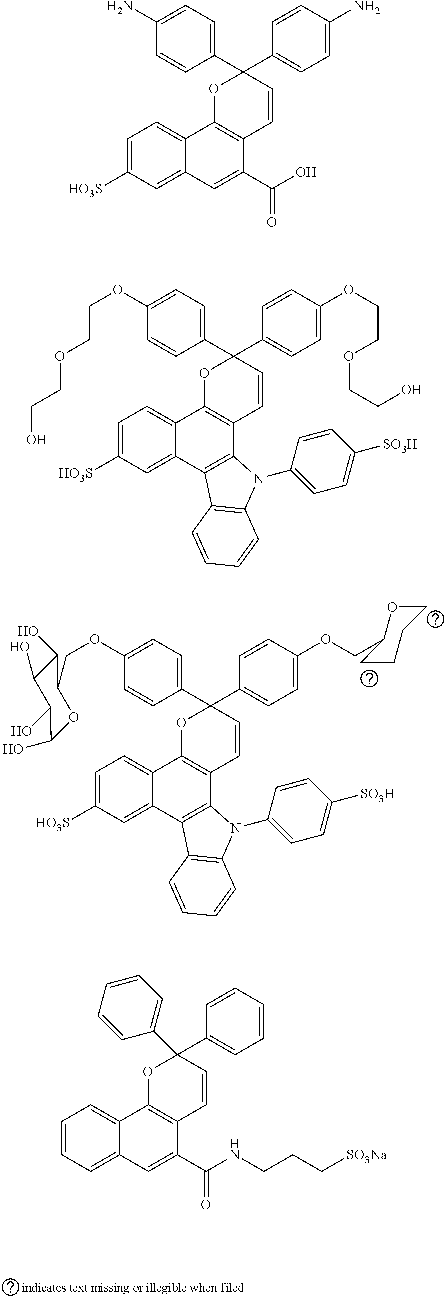

In other embodiment, the conformation of the molecule of the photochromic naphthopyran compound can be changed to ring-open form upon UV or visible light irradiation, and can be thermodynamically changed backward to the ring-close form:

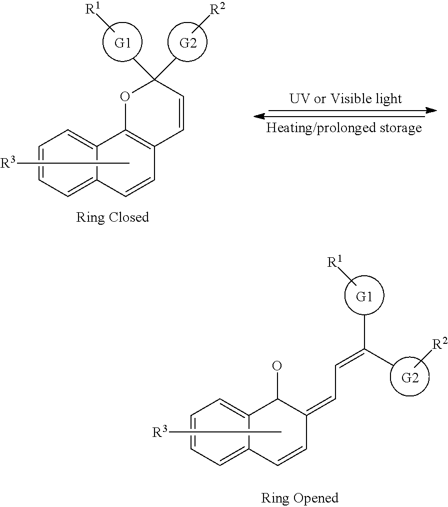

In another embodiment, the water solubility of the photochromic naphthopyran compound is great than or equal to 10 mg L⁻¹.

In other embodiment, there is provided a photochromic mixture comprising at least one of the photochromic naphthopyran compound, where the weight percent of the photochromic naphthopyran compound in the mixture is greater than 0.01%.

In another embodiment, said photochromic mixture has a water solubility greater than or equal to 10 mg L⁻¹.

In yet another embodiment, said photochromic mixture displays photochromic property upon UV or visible light irradiation, and wherein the photochromism is thermodynamically reversible.

In other embodiment, there is provided a photochromic polymer thin film comprising one or more polymers and/or resins and at least one the photochromic naphthopyran compound, wherein the one or more polymers and/or resins is/are selected from a wide range of commodity polymers, engineering polymers, high-performance polymers and polyelectrolyte with an amount of approximately 10 to 70 weight percent.

In another embodiment, said photochromic polymer thin film further comprises one or more additives selected from waxes, dispersing powder, calcium carbonate, glycerol, ethoxylated or propoxylated fatty alcohols, inorganic metal/metal oxide nanoparticles with an amount from approximately 0 to 5 weight percent.

In yet another embodiment, said photochromic polymer thin film is deposited on substrates including plastic, glass, ceramics, metal or metal alloys, wood or paper.

In still another embodiment, said photochromic polymer thin film is produced by solution-based thin-film deposition techniques including spin-coating, dip-coating, spray-coating and drop-casting methods.

### BRIEF DESCRIPTION OF THE DRAWINGS

In the following detailed description, reference is made to the accompanying figures, depicting exemplary, non-limiting and non-exhaustive embodiments of the invention. So that the manner in which the above recited features of the present invention can be understood in detail, a more particular description of the invention, briefly summarized above, can be had by reference to the embodiments, some of which are illustrated in the appended figures. It should be noted, however, that the figures illustrate only typical embodiments of this invention and are therefore not to be considered limiting of its scope, for the invention can admit to other equally effective embodiments.

FIG. 1 shows the stimulated UV/visible light absorption spectrum of NAP1W (black line) and its ring open form NAP1-RO (red line).

FIG. 2 shows the stimulated UV/visible light absorption spectrum of NAP2W (black line) and its ring open form NAP2-RO (red line).

FIG. 3 shows the stimulated UV/visible light absorption spectrum of NAP3W (black line) and its ring open form NAP3-RO (red line).

FIG. 4 shows the stimulated UV/visible light absorption spectrum of NAP4W (black line) and its ring open form NAP4-RO (red line).

FIG. 5 shows the stimulated UV/visible light absorption spectrum of NAP5W (black line) and its ring open form NAP5-RO (red line).

FIG. 6 shows the stimulated UV/visible light absorption spectrum of NAP6W (black line) and its ring open form NAP6-RO (red line).

FIG. 7 shows the stimulated UV/visible light absorption spectrum of NAP7W (black line) and its ring open form NAP7-RO (red line).

FIG. 8 shows the stimulated UV/visible light absorption spectrum of NAP8W (black line) and its ring open form NAP8-RO (red line).

### DEFINITIONS

### Alkyl

The term “alkyl” as used herein includes reference to an unbranched or branched chain alkyl moiety having 1, 2, 3, 4, 5, 6, 7, 8, 9, 10, 11, 12, 13, 14, 15, 16, 17, 18, 19 or 20 carbon atoms.

### Alkoxy

The terms “alkoxy” as used herein include reference to —O-alkyl, wherein alkyl is unbranched or branched chain and comprises 1, 2, 3, 4, 5 or 6 carbon atoms. In one class of embodiments, alkoxy has 1, 2, 3 or 4 carbon atoms. This term includes reference to groups such as methoxy, ethoxy, propoxy, isopropoxy, butoxy, tert- butoxy, pentoxy, hexoxy and the like.

### Aryl

The term “aryl” as used herein includes reference to an aromatic ring system comprising 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

### Heteroaryl

The term “heteroaryl” as used herein includes reference to an aromatic heterocyclic ring system having 5, 6, 7, 8, 9, 10, 11, 12, 13, 14, 15 or 16 ring atoms, at least one of which is selected from nitrogen, oxygen and sulphur. The group may be a polycyclic ring system, having two or more rings, at least one of which is aromatic, but is more often monocyclic. The ring or ring system may be substituted with one or more hydrocarbyl groups. This term includes reference to groups such as pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl, pteridinyl and the like.

### Halogen

The term “halogen” as used herein includes F, Cl, Br or I.

### DETAILED DESCRIPTION

In order to create photochromic compounds that have color-changing properties in response to visible light activation, thermodynamic reversibility of the photochromism and water solubility, various photochromic naphthopyrans compounds that color change in response to UV light are provided. Upon introducing water-solubilizing groups in the naphthopyran system, including modified sulfonate, hydroxyl and amino groups, water solubility of the naphthopyrans compounds is enhanced while the photochromism properties retained.

The present invention provides a water-soluble photochromic naphthopyran compound having a general structural formula represented by Formula (1):

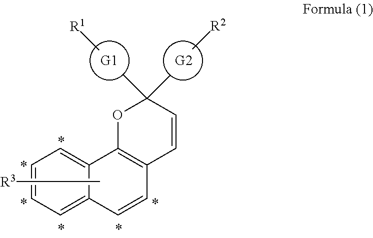

G1 is an aromatic group containing 5 to 20 ring atoms, a heteroaromatic group containing 5 to 20 ring atoms, or a non-aromatic ring system containing 5 to 20 ring atoms; G1 has a substituent R¹, and the R¹ is the same or different in multiple occurrences.
G2 is an aromatic group containing 5 to 20 ring atoms, a heteroaromatic group containing 5 to 20 ring atoms, or a non-aromatic ring system containing 5 to 20 ring atoms; G2 has a substituent R², and the R² is the same or different in multiple occurrences.
The symbol “*” represent possible bonding locations of Formula (1) with following chemical formulae:

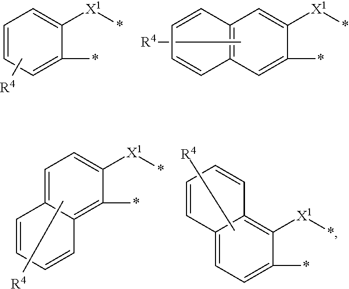

In these formulae, X¹ is CR⁵CR⁶, O, S, NR⁷, Se, S(═O)₂, C═O, S═O, or PR⁸.
R¹, R², R³, R⁴, R⁵, R⁶, R⁷, R⁸ are independently hydrogen, deuterium, a halogen atom, a water-solubilizing group, —OCH₃, —NO₂, —C≡N, —N≡C, a linear alkyl group containing 1 to 20 carbon atoms, a branched alkyl group containing 1 to 20 carbon atoms, or a linear alkenyl group containing 1 to 20 carbon atoms, a branched alkenyl group containing 1 to 20 carbon atoms, an alkane ether group containing 1 to 20 carbon atoms, an aromatic containing 1 to 20 carbon atoms, a heteroaromatic containing 1 to 20 carbon atoms or a non-aromatic ring system containing 1 to 20 carbon atoms.
R¹, R², R³, R⁴, R⁵, R⁶, R⁷, R⁸ independently contain at least one water-solubilizing group selected from —SO₃H, —OH, —NCₙH₂ₙ₊₁H⁺, —N(CₙH₂ₙ₊₁)₃⁺, —SO₃Na, —SO₃H, —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin.

G1 and G2 in Formula (1) of the photochromic napthopyran compound can be one of the following formulae:

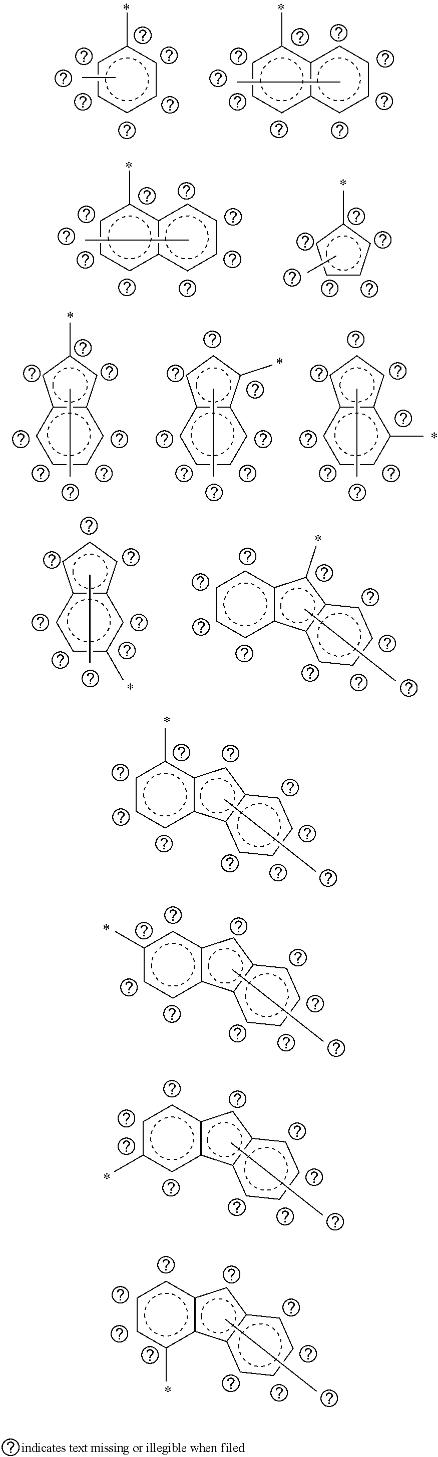

In these formulae, # represents the bonding location in Formula (1).
Z¹-Z⁹ are independently or jointly carbon, nitrogen, oxygen, silicon, boron, sulfur or phosphorus atom.
R⁹ are independently or jointly hydrogen, deuterium, a halogen atom, —OCH₃, —NO₂, —C≡N, —N≡C, —NPh₂, N-carbazole, dibenzofuran, dibenzothiophene, —SO₃H, —OH, —NCₙH₂ₙ₊₁H⁺, —N(CₙH₂ₙ₊₁)₃⁺, —SO₃Na, —OSO₃H, —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin or a linear alkyl group containing 1 to 20 carbon atoms, a branched alkyl group containing 1 to 20 carbon atoms, and a linear alkenyl group containing 1 to 20 carbon atoms, a branched alkenyl group containing 1 to 20 carbon atoms, an alkane ether group containing 1 to 20 carbon atoms, an aromatic containing 1 to 20 carbon atoms, a heteroaromatic containing 1 to 20 carbon atoms or a non-aromatic ring system containing 1 to 20 carbon atoms.

A photochromic printing ink may be made from the water-soluble photochromic naphthopyran compounds of the present invention. The ink may be an offset printing ink, screen printing ink, or inkjet printing ink. The ink compositions may include a naphthopyran compound of Formula 1 in an amount of 0.1 to 15 weight percent; one or more polymers/resins is included in an amount of approximately 0 to 50 weight percent; one or more additives in an amount from approximately 0 to 8 weight percent; and a solvent in an amount of approximately 50 to 99.9 weight percent.

In a particular ink, the photochromic naphthopyran compound of Formula 1 is included in the ink composition is in an amount of approximately 0.5 to 15 weight percent, and the ink composition further includes the additives in an amount of approximately 0.5 to 5 weight percent selected from one or more of waxes, dispersing powder, calcium carbonate; the solvent in an amount of approximately 50 to 70% weight percent selected from one or more of dichloromethane, esters, ketones, soybean oil, mineral oil, tung oil and linseed oil; the one or more resins selected from resin or rosin modified phenolic resin, in an amount of 30 to 50%.

In another ink composition, the photochromic naphthopyran compound in the screen printing ink composition is included in an amount of approximately 0.1 to 3 weight percent, and the screen-printing ink further includes the solvent in an amount of approximately 50 to 70% weight percent selected from one or more of cyclohexanone, xylene and tung oil; the one or more resins are selected from synthetic resins such as phenolic resins in an amount of 30 to 50%.

In another ink composition, the photochromic naphthopyran compound of Formula 1 is included is in an amount of approximately 0.1 to 6 weight percent, and the ink composition further includes the solvent in an amount of approximately 94 to 99.9% weight percent selected from one or more of ethyl acetate, 2-butanone and diethylene glycol monoethyl ether acetate.

In another aspect, the present invention provides a photochromic composition that has two or more photochromic naphthopyran-based components with different thermal decay rate constants (k). By using multiple naphthopyran-based components, a controlled time and color may be engineered in a product such as an ink or polymeric article such as a thin film or solid polymer article. In particular, a gradual color-changing effect may be realized using the photochromic compositions of the present invention. Typical solid articles include windows, lenses, and optical filters which may be formed by incorporating the photochromic composition or by coating using a thin film or ink including the photochromic composition. Further applications include novelty items, toys, and decorative articles that exhibit color changing properties.

The compounds of the present invention may be used in a large number of applications. In one aspect, the composition is used as a part of a photochromic ink. There are a number of types of photochromic inks that may include the photochromic composition such as offset inks, screen printing inks, and inkjet printing inks. These different types of inks have different requirements in terms of selected solvents, polymers, and additives, depending upon the required properties for the ink such as viscosity, desired thickness of the deposited ink, resolution, etc. In general, the photochromic composition is used in the ink in an amount of approximately 0.5 to 20 weight percent. Advantageously, the water-soluble properties of the naphthopyran compounds means that they can be used in water-soluble applications such as water-soluble inks.

The ink further includes one or more polymers/resins in an amount of approximately 3 to 30 weight percent, one or more additives in an amount from approximately 0 to 8 weight percent; and a solvent in an amount of approximately 70 to 96.5 weight percent.

The one or more polymers/resins may be selected from cellulose, starch, rubber, shellac, rosin modified phenolic resin. The one or more additives is/are selected from waxes, dispersing powder, calcium carbonate, glycerol, ethoxylated or propoxylated fatty alcohols. The solvent may be selected from one or more of dichloromethane, esters, ketones, soybean oil, mineral oil, tung oil, linseed oil, hexane, toluene, xylene, dichloromethane, chloroform, dichlorobenzene, esters, ketones, and water.

The water-soluble naphthopyran photochromic compound may also be incorporated into a polymeric material that includes the photochromic compound. The photochromic polymeric material may include one or more polymers selected from polyethylene, polypropylene, polystyrene, polyvinyl chloride, polyamides, polyethylene terephthalate, polytetrafluoroethylene, poly(methyl methacrylate), polyphenylene sulfide, and polyether ether ketone, or rosin-modified phenolic resin. Photochromic polymer articles can be formed through conventional polymer processing such as extrusion, molding, casting, etc.

Various arbitrary selection of color changes may be obtained through the mixing of two photochromic naphthopyran compounds with different half-lives or different ratios, described above.

In other aspect, the photochromic naphthopyran compounds can be dissolved into any water-based matrix where water is the solubilizing medium to form a photochromic material for further applications.

In another aspect, the naphthopyran compounds may be used in thin films; the thin films may be optionally deposited on substrates such as substrates including plastic, glass, ceramics, metal or metal alloys, wood or paper.

As thin films, the compounds may be used for security features in products. A photochromic polymer thin film may include one or more polymers and/or resins and at least one photochromic naphthopyran compound of Formula 1. The one or more polymers and/or resins is/are selected from commodity polymers, engineering polymers, high-performance polymers and polyelectrolytes with an amount of approximately 10 to 70 weight percent. The thin film may include additives such as waxes, dispersing powder, calcium carbonate, glycerol, ethoxylated or propoxylated fatty alcohols, inorganic metal/metal oxide nanoparticles with an amount from approximately 0 to 5 weight percent. The mixtures may have a water solubility greater than or equal to 10 mg L⁻¹. and have photochromism responsive to UV radiation or visible light. Heating may be used to reverse the color change. The absorption peak in UV/vis absorption spectrum ranges from 100 nms to 1500 nm.

Solution-based thin-film deposition techniques such as spin-coating, dip-coating, spray-coating and drop-casting may be used to produce the photochromic polymer thin film.

### EXAMPLES

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### 1. Selection of Examples of Water-Soluble Naphthopyrans

Photochromic naphthopyran compounds of Formula (1) can include, but are not limited to, those compounds listed in Table 1.

TABLE 1
Water-soluble photochromic naphthopyran compounds

| Compound | Structure |
|---|---|
| NAP1W | 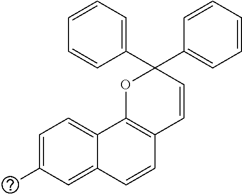 [SMILES: Cc1ccc2c3c(ccc2c1)C=CC(c1ccccc1)(c1ccccc1)O3] |
| NAP2W | 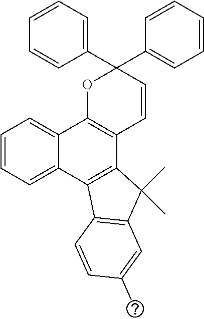 [SMILES: Cc1ccc2c(c1)C(C)(C)c1c3c(c4ccccc4c1-2)OC(c1ccccc1)(c1ccccc1)C=C3] |
| NAP3W | 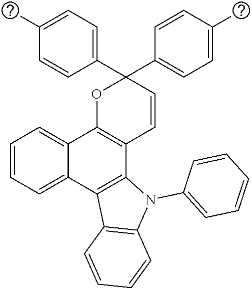 [SMILES: Cc1ccc(C2(c3ccc(C)cc3)C=Cc3c(c4ccccc4c4c5ccccc5n(-c5ccccc5)c34)O2)cc1] |
| NAP4W | 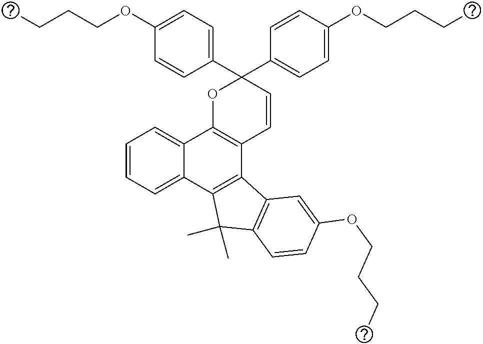 [SMILES: CCCCOc1ccc(C2(c3ccc(OCCCC)cc3)C=Cc3c4c(c5ccccc5c3O2)C(C)(C)c2ccc(OCCCC)cc2-4)cc1] |
| NAP5W | 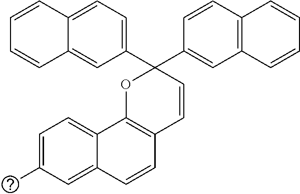 [SMILES: Cc1ccc2c3c(ccc2c1)C=CC(c1ccc2ccccc2c1)(c1ccc2ccccc2c1)O3] |
| NAP6W | 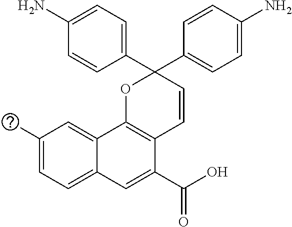 [SMILES: Cc1ccc2cc(C(=O)O)c3c(c2c1)OC(c1ccc(N)cc1)(c1ccc(N)cc1)C=C3] |
| NAP7W | 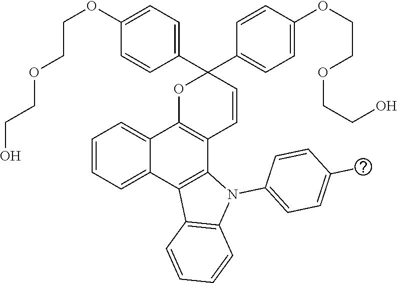 [SMILES: Cc1ccc(-n2c3ccccc3c3c4ccccc4c4c(c32)C=CC(c2ccc(OCCOCCO)cc2)(c2ccc(OCCOCCO)cc2)O4)cc1] |
| NAP8W | 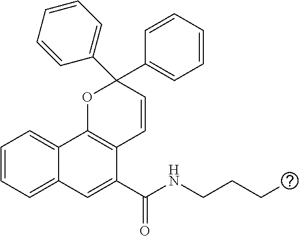 [SMILES: CCCCNC(=O)c1cc2ccccc2c2c1C=CC(c1ccccc1)(c1ccccc1)O2] |

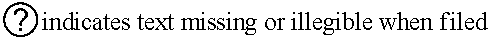

The energy levels of the metal organic complexes can be obtained by quantum calculations, for example, by using Time Dependent-Density Functional Theory (TD-DFT) through Gaussian 16W (Gaussian Inc.). Firstly, the molecular geometry is optimized by DFT method (Density Functional Theory) “Ground State/DFT/Default Spin/B3LYP” with basic set “6-31G d” (Charge 0/Spin Singlet), and then energy of the optimized structure is calculated by TD-DFT method “TD-SCF/DFT/Default Spin/B3PW91” with basic set “6-31G d” (Charge 0/Spin Singlet). The results calculated by Gaussian 16W are used directly without calibration. The calculated S1, S2, S3, S4, S5, S6 of the selected examples of water-soluble naphthopyrans were shown in Table 2 and that of their corresponding ring-opened (RO) form were shown in Table 3.

TABLE 2

| | Singlet state [nm] | | | | | |
|---|---|---|---|---|---|---|
| Materials | S1 | S2 | S3 | S4 | S5 | S6 |
| NAP1W | 359.47 | 317.57 | 285.43 | 275.80 | 270.70 | 269.75 |
| NAP2W | 377.47 | 342.64 | 305.45 | 289.38 | 287.43 | 279.89 |
| NAP3W | 396.20 | 367.34 | 358.92 | 355.36 | 332.01 | 322.14 |
| NAP4W | 366.58 | 342.90 | 309.08 | 303.65 | 295.61 | 284.06 |
| NAP5W | 362.05 | 337.58 | 332.62 | 319.08 | 311.26 | 303.71 |
| NAP6W | 414.58 | 390.92 | 385.27 | 369.55 | 349.93 | 336.50 |
| NAP7W | 408.55 | 374.62 | 360.77 | 342.63 | 337.86 | 318.65 |
| NAP8W | 360.34 | 335.44 | 272.54 | 272.20 | 268.22 | 266.58 |

TABLE 3

| | Singlet state [nm] | | | | | |
|---|---|---|---|---|---|---|
| Materials | S1 | S2 | S3 | S4 | S5 | S6 |
| NAP1-RO | 486.61 | 465.07 | 374.61 | 343.48 | 335.42 | 333.22 |
| NAP2-RO | 530.30 | 465.11 | 384.84 | 352.87 | 339.23 | 336.84 |
| NAP3-RO | 610.86 | 473.17 | 435.32 | 376.50 | 365.07 | 352.38 |
| NAP4-RO | 539.44 | 448.50 | 433.69 | 395.70 | 362.26 | 345.38 |
| NAP5-RO | 508.31 | 469.88 | 445.18 | 434.60 | 380.43 | 347.83 |
| NAP6-RO | 518.68 | 464.81 | 432.85 | 384.47 | 378.59 | 322.85 |
| NAP7-RO | 546.11 | 452.40 | 429.83 | 424.67 | 390.69 | 365.97 |
| NAP8-RO | 472.33 | 462.94 | 368.63 | 344.93 | 338.10 | 333.57 |

### 2. Synthesis Examples of Selected Water-Soluble Naphthopyrans

### Synthesis of NAP1W

A schematic diagram of the synthesis of NAP1W is as below:

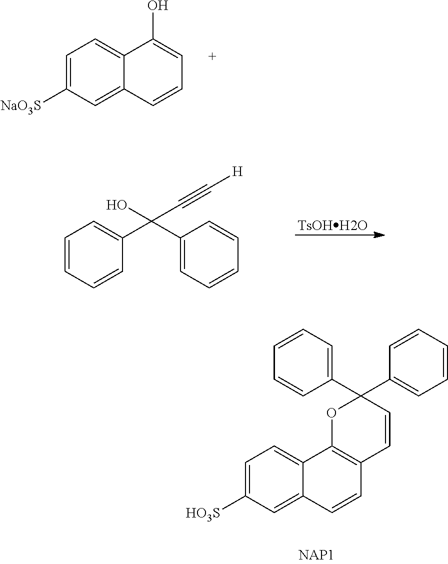

In a 250 mL round-bottomed flask equipped with a magnetic stirrer, sodium 6-hydroxynaphthalene-2-sulfonate (CAS: 135-76-2) (11.82 g, 48 mmol), 1,1-diphenyl-2-propyn-1-01 (CAS: 3923-52-2) (10 g, 48 mmol), p-toluenesulfonic acid monohydrate (0.46 g, 2.4 mmol) and THF (30 ml) were added. The mixture was heated to 120° C. overnight. The mixture was evaporated to dryness and purified by reverse phase column chromatography using water as eluent, followed by washing with DCM to give red solid. Yield: 6.5 g (51%). Negative ESI-MS: ion clusters at m/z 413 [M-H]⁻.

### Synthesis of NAP2W

A schematic diagram of the synthesis of NAP2W is as below:

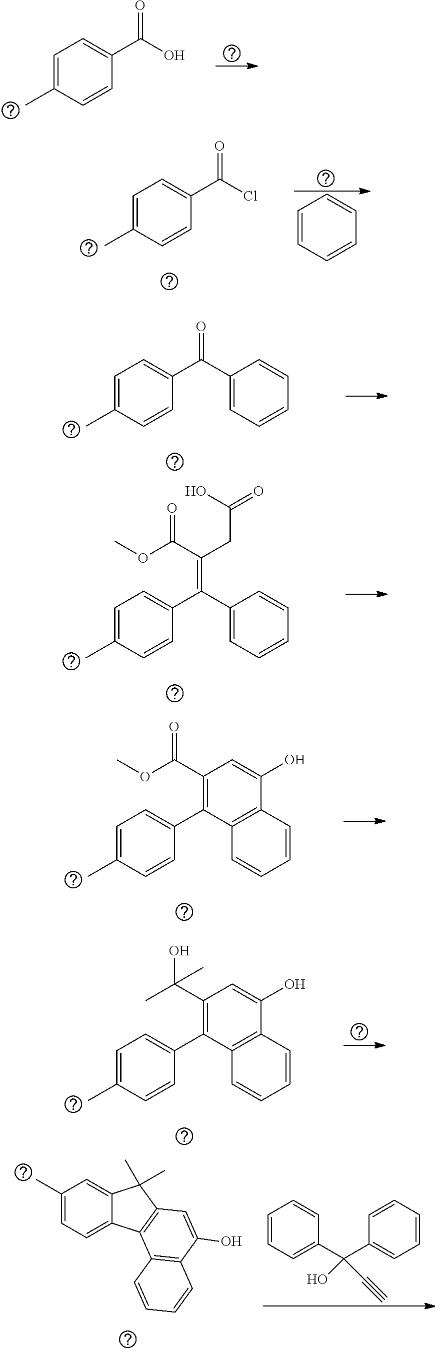

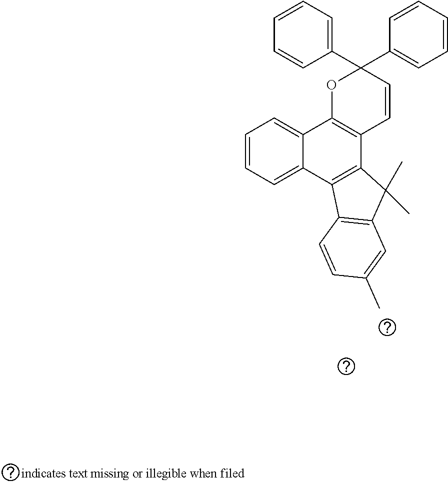 [SMILES: Cc1ccc2c(c1)C(C)(C)c1c3c(c4ccccc4c1-2)OC(c1ccccc1)(c1ccccc1)C=C3]

To obtain NAP2-a, heat a mixture of 4-sulfobenzoic monopotassium salt (CAS: 5399-63-3) (50 g, 208 mmol) in freshly distilled thionyl chloride (CAS: 7719-09-7) (50 g. 420 mmol) to reflux in a 250 mL round-bottomed flask equipped with a magnetic stirrer for 2 hours. After that, the reaction was allowed to cool to room temperature and evaporate under vacuum to dryness to give white solid. Yield: 43.6 g (95%). Negative ESI-MS: ion clusters at m/z 218 [M-H]⁻.

To obtain NAP2-b, in a 250 mL round-bottomed flask equipped with a magnetic stirrer, NAP2-a (40 g, 181 mmol) and dry benzene (100 mL) were added. Then, the mixture was stirred for 5 minutes followed by addition of aluminum chloride (AlCl₃, CAS: 7446-70-0) (120 g, 906 mmol) to the mixture slowly. Then the reaction was allowed to reflux for 4 hours. The reaction was evaporated via vacuum to remove unreacted benzene and then purified by reverse phase column chromatography using water as eluent, followed by washing with DCM to give white solid. Yield: 35.4 g (74%). Negative ESI-MS: ion clusters at m/z 262 [M-H]⁻.

To obtain NAP2-c, in a 250 mL round-bottomed flask equipped with a magnetic stirrer in ice water bath, NAP2-b (11.27 g, 43 mmol) and diglyme (100 ml) were added. Then, dimethyl succinate (CAS: 106-65-0) (7.4 ml, 56 mmol) and potassium tert-butoxide (5.5 g, 49 mmol) (separated in 3 portions) were added. The mixture was stirred at room temperature for 4 hours. Then, the mixture was evaporated to dryness via vacuum and purified by reverse phase column chromatography using water as eluent, followed by washing with DCM to give brown solid. Yield: 14.7 g (91%). Negative ESI-MS: ion clusters at m/z 375 [M-H]⁻.

To obtain NAP2-d, in a 100 mL round-bottomed flask equipped with a magnetic stirrer, NAP2-c (10.16 g, 27 mmol) was mixed with acetic anhydride (45 ml) and heated to reflux for 3 hours. Then, the mixture was evaporated to dryness via vacuum and purified by reverse phase column chromatography using water as eluent, followed by washing with DCM to give beige solid. Yield: 4.74 g (49%). Negative ESI-MS: ion clusters at m/z 357 [M-H]⁻.

To obtain NAP2-e, methylmagnesium bromide (CAS: 75-16-1) in THF (1.4 M, 19 ml) was added dropwise under nitrogen to a THF solution (40 ml) of NAP2-d (3.0 g, 8.4 mmol) in ice water bath. The mixture was warmed to room temperature and stirred for 12 hours. Then, the mixture was evaporated to dryness via vacuum and purified by reverse phase column chromatography using water as eluent, followed by washing with DCM to give red solid. Yield: 0.78 g (26%). Negative ESI-MS: ion clusters at m/z 357 [M-H]⁻.

To obtain NAP2-f, in a 100 mL round-bottomed flask equipped with a magnetic stirrer, NAP2-e (0.65 g, 1.9 mmol), p-toluenesulfonic acid monohydrate (0.345 g, 1.9 mmol) and diglyme (40 ml) were added. The mixture was heated to reflux for 8 hours. Then, the mixture was evaporated to dryness via vacuum and purified by reverse phase column chromatography using water as eluent, followed by washing with DCM to give white solid. Yield: 0.463 g (75%). Negative ESI-MS: ion clusters at m/z 339 [M-H]⁻.

To obtain the final product of NAP2W, in a 100 mL round-bottomed flask equipped with a magnetic stirrer, NAP2-f (0.61 g, 1.8 mmol), 1,1-diphenyl-2-propyn-1-ol (0.37 g, 1.8 mmol), p-toluenesulfonic acid monohydrate (0.342 g, 1.8 mmol) and diglyme (30 ml) were added. The mixture was heated to reflux for 12 hours. Then, the mixture was evaporated to dryness via vacuum and purified by reverse phase column chromatography using water as eluent, followed by washing with DCM to give pale yellow product. Yield: 0.42 g (44%). Negative ESI-MS: ion clusters at m/z 529 [M-H]⁻.

### Synthesis of NAP3W

A schematic diagram of the synthesis of NAP3W is as below:

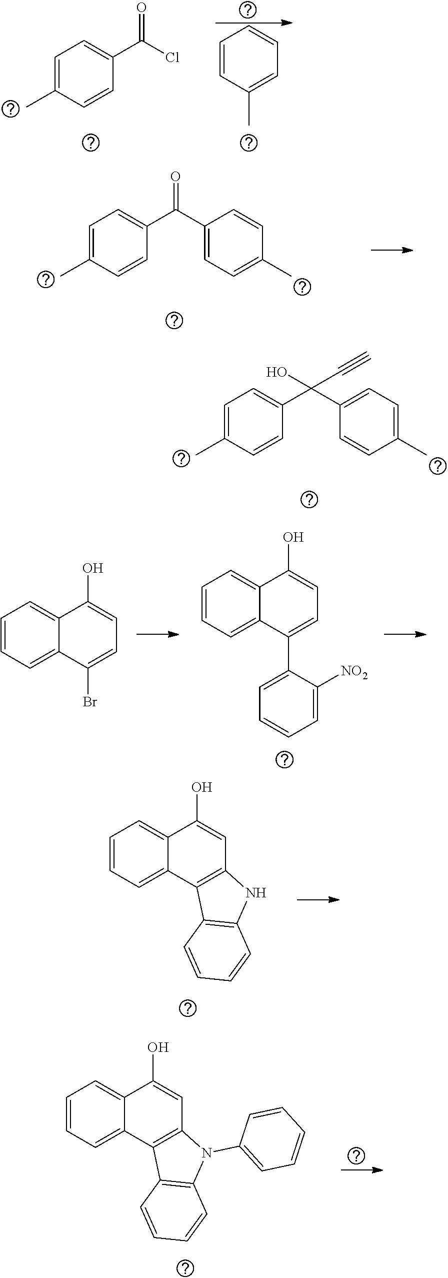

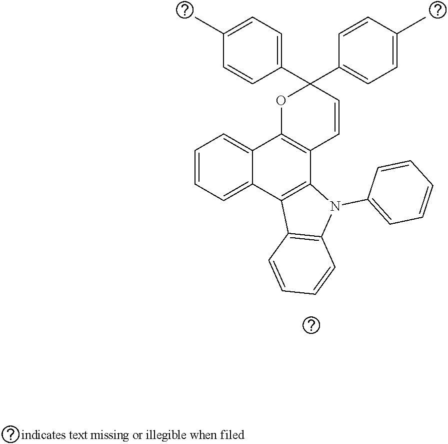 [SMILES: Cc1ccc(C2(c3ccc(C)cc3)C=Cc3c(c4ccccc4c4c5ccccc5n(-c5ccccc5)c34)O2)cc1]

To obtain NAP3-a, in a 1000 mL round-bottomed flask equipped with a magnetic stirrer, add benzenesulfonic acid (CAS: 98-11-3) (286.28 g, 1810 mmol) and NAP2-a (80 g, 362 mmol) to the flask and then use THF (600 mL) to completely dissolve the solid. Then, the mixture was stirred for 5 minutes followed by addition of aluminum chloride (AlCl₃, CAS: 7446-70-0) (240 g, 1810 mmol) to the mixture slowly. Then the reaction was allowed to reflux for 4 hours. The reaction was evaporated via vacuum to remove solvent and then purified by reverse phase column chromatography using water as eluent, followed by washing with DCM to give white solid. Yield: 84.4 g (68%). Negative ESI-MS: ion clusters at m/z 169 [M-2H]²⁻ and 340 [M-H]⁻.

To obtain NAP3-b, sodium acetylide suspension (18 wt %) in THF solution was added dropwise under nitrogen to a THF solution (200 ml) of NAP3-a (80.8 g, 236 mmol) in ice water bath. The mixture was warmed to room temperature and stirred for 6 hours. Then, the mixture was evaporated to dryness via vacuum and purified by reverse phase column chromatography using water as eluent, followed by washing with DCM to give pale yellow oil. Yield: 31.3 g (36%). Negative ESI-MS: ion clusters at m/z 183 [M-2H]²⁻ and 367 [M-H]⁻.

To obtain NAP3-c, in a 500 mL round-bottomed flask equipped with a magnetic stirrer, 4-bromonaphthalene-1-ol (CAS: 571-57-3) (20 g, 89.7 mmol), 2-nitrophenylboronic acid (CAS: 5570-19-4) (15.7 g, 94.1 mmol), tetrakis(triphenylphosphine)palladium(0) (Pd(PPh₃)₄, CAS: 14221-01-3) (10.36 g, 9.0 mmol) and potassium carbonate (49.5 g, 359 mmol) were mixed, then 250 mL of a mixture of 1,4-dioxane and water in a ratio of 3:1 was added. The mixture reacted under stirring at 90° C. for 12 hours, After the reaction was completed, then cooled to room temperature. The solvent was dried by rotary evaporation, and then the mixture was washed with dichloromethane and water. The organic layer was collected and dried with magnesium sulfate, and was dried by rotary evaporation, followed by the column chromatography to obtain a white solid. Yield: 15.46 g (65%). Positive EI-MS: ion clusters at m/z 265 [M]⁺.

To obtain NAP3-d, in a 250 mL round-bottomed flask equipped with a magnetic stirrer, NAP3-c (15 g, 56.6 mmol) and triphenyl phosphine (PPh₃, CAS: 603-35-0) (29.7 g, 113 mmol) were added and then o-dichlorobenzene (150 mL) was added to dissolve the solid. The reaction was allowed to heat at 200° C. for 24 hours. After the reaction complete, the reaction was cooled down to room temperature and remove the solvent by vacuum, followed by extraction with DCM and water. The organic layer was collected and then dried by magnesium sulfate. The mixture was then purified by column chromatography and obtain the white solid. Yield: 9.64 g (73%). Positive EI-MS: ion clusters at m/z 233 [M]⁺.

To obtain NAP3-e, in a 250 mL round-bottomed flask equipped with a magnetic stirrer, NAP3-d (9 g, 38.6 mmol) was added and dissolve in THF (100 mL). And then THF suspension (50 mL) of sodium hydride (4.6 g, 193 mmol) was slowly added at 0° C., followed by stirring at 0° C. for 10 minutes. Fluorobenzene (CAS: 462-06-6) (3.9 g, 40.5 mmol) was then slowly added into the reaction mixture. After the addition, the reaction was allowed to stir at room temperature for 4 hours. After the reaction complete, ethanol was added to quench the reaction. The solvent was removed by vacuum, followed by extraction with DCM and water. The organic layer was collected and then dried by magnesium sulfate. The mixture was then purified by column chromatography and obtain the white solid. Yield: 10.5 g (88%). Positive EI-MS: ion clusters at m/z 309 [M]⁺.

To obtain the final product of NAP3W, in a 250 mL round-bottomed flask equipped with a magnetic stirrer, NAP3-e (10 g, 32.3 mmol), NAP3-b (11.9 g, 32.3 mmol), p-toluenesulfonic acid monohydrate (0.684 g, 3.6 mmol) and diglyme (150 ml) were added. The mixture was heated to reflux for 12 hours. Then, the mixture was evaporated to dryness via vacuum and purified by reverse phase column chromatography using water as eluent, followed by washing with DCM to give pale yellow product. Yield: 6.83 g (32%). Negative ESI-MS: ion clusters at m/z 328 [M-2H]²⁻ and 658 [M-H]⁻.

### Synthesis of NAP4W

A schematic diagram of the synthesis of NAP4W is as below:

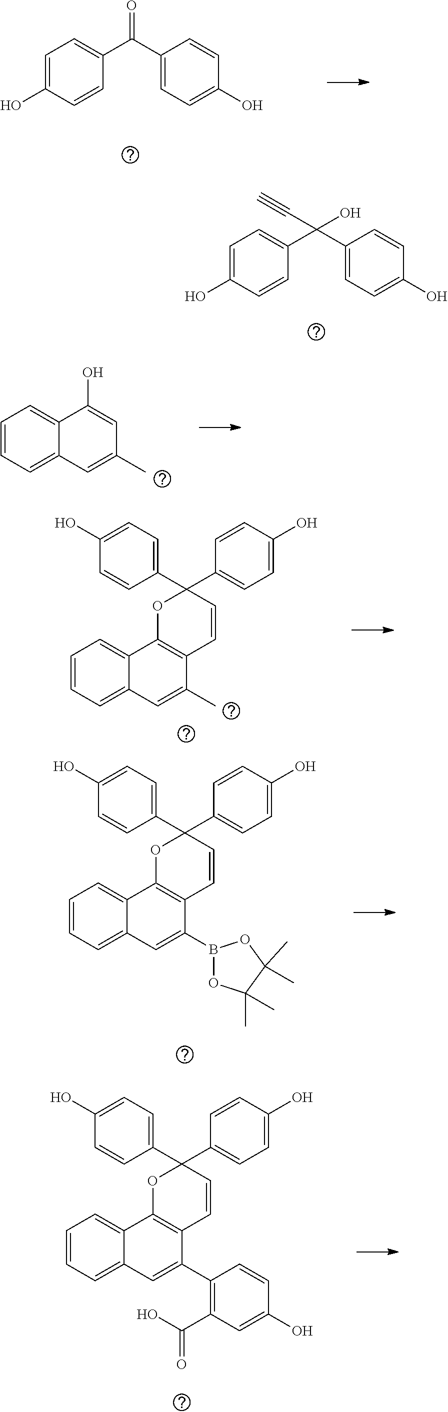 [SMILES: O=C(c1ccc(O)cc1)c1ccc(O)cc1 ; C#CC(O)(c1ccc(O)cc1)c1ccc(O)cc1 ; Cc1cc(O)c2ccccc2c1 ; Cc1cc2ccccc2c2c1C=CC(c1ccc(O)cc1)(c1ccc(O)cc1)O2 ; CC1(C)OB(c2cc3ccccc3c3c2C=CC(c2ccc(O)cc2)(c2ccc(O)cc2)O3)OC1(C)C ; O=C(O)c1cc(O)ccc1-c1cc2ccccc2c2c1C=CC(c1ccc(O)cc1)(c1ccc(O)cc1)O2]

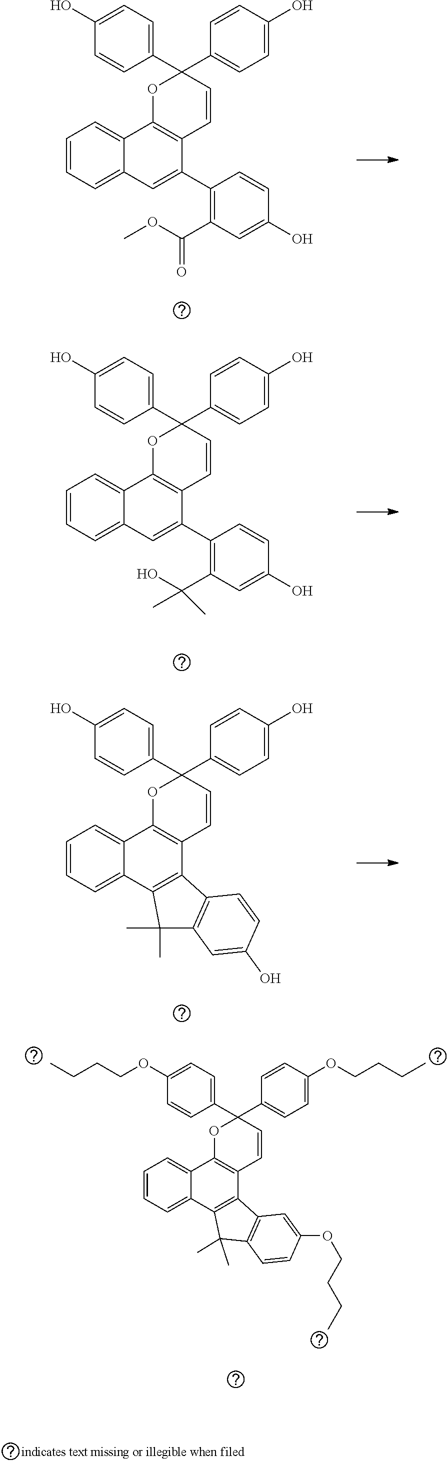 [SMILES: COC(=O)c1cc(O)ccc1-c1cc2ccccc2c2c1C=CC(c1ccc(O)cc1)(c1ccc(O)cc1)O2 ; CC(C)(O)c1cc(O)ccc1-c1cc2ccccc2c2c1C=CC(c1ccc(O)cc1)(c1ccc(O)cc1)O2 ; CC1(C)c2cc(O)ccc2-c2c3c(c4ccccc4c21)OC(c1ccc(O)cc1)(c1ccc(O)cc1)C=C3 ; CCCCOc1ccc(C2(c3ccc(OCCCC)cc3)C=Cc3c4c(c5ccccc5c3O2)C(C)(C)c2ccc(OCCCC)cc2-4)cc1]

To obtain NAP4-a, to a THF solution (200 mL) of 4,4′-dihydroxybenzophenone (CAS: 611-99-4) (51 g, 236 mmol) in ice water bath, sodium acetylide suspension (18 wt %) in THF solution was added dropwise under nitrogen. The mixture was warmed to room temperature and stirred for 6 hours. Then, the mixture was evaporated to dryness via vacuum and purified by reverse phase column chromatography using water as eluent, followed by washing with DCM to give pale yellow oil. Yield: 32.5 g (36%). Positive EI-MS: ion clusters at m/z 240 [M]⁺.

To obtain NAP4-b, in a 250 mL round-bottomed flask equipped with a magnetic stirrer, 3-bromo-1-hydroxynaphthalene (CAS: 90767-17-2) (7.2 g, 32.3 mmol), NAP4-a (7.75 g, 32.3 mmol), p-toluenesulfonic acid monohydrate (0.684 g, 3.6 mmol) and toluene (100 ml) were added. The mixture was heated to reflux for 12 hours. Then, the mixture was evaporated to dryness via vacuum and purified by column chromatography using DCM/MeOH (10:1 v/v) as eluent to obtain the white solid. Yield: 6.76 g (47%). Positive EI-MS: ion clusters at m/z 445 [M]⁺.

To obtain NAP4-c, in a 500 mL round-bottomed flask equipped with a magnetic stirrer, NAP4-b (6 g, 13.5 mmol), bis(pinacolato)diboron (CAS: 73183-34-3) (4.45 g , 17.5 mmol), 1,1′-bis(diphenylphosphino)ferrocene]dichloropalladium(II) (Pd(dppf)₂Cl₂, CAS: 72287-26-4) (0.98 g, 1.35 mmol), potassium acetate (5.3 g, 54 mmol) and 1,4-dioxane:water (3:1 v/v) (250 mL) were added. The mixture was stirred and heated to 90° C. for 12 hours. The reaction was then allowed to cool down to room temperature, followed by evaporation of solvent to dryness via vacuum and then extraction by using DCM and H₂O. The organic layer was collected and dried by magnesium sulfate. The crude mixture was then purified by column chromatography using DCM/MeOH (10:1 v/v) as eluent to obtain the white solid. Yield: 5.24 g (79%). Positive EI-MS: ion clusters at m/z 492 [M]⁺.

To obtain NAP4-d, in a 250 mL round-bottomed flask equipped with a magnetic stirrer, 2-bromo-5-hydroxybenzoic acid (CAS: 58380-11-3) (2.2 g, 10.1 mmol), NAP4-c (5 g, 10.1 mmol), Pd(PPh₃)₄ (1.16 g, 1.01 mmol), and potassium carbonate (7.0 g, 50.5 mmol) and 150 mL mixture of 1,4-dioxane and water in a ratio of 3:1 were added. The mixture was reacted under stirring at 90° C. for 12 hours. After the reaction was completed, it was allowed to cool down to room temperature. Then, the solvent was dried by rotary evaporation, and the crude product was extracted with dichloromethane and water. The organic layer was collected and dried with magnesium sulfate, followed by the purification by column chromatography to obtain a white solid. Yield: 1.37 g (61%). Positive EI-MS: ion clusters at m/z 502 [M]⁺.

To obtain NAP4-e, a solid NAP4-d (1 g, 2 mmol) was added into a dry two-neck flask, then 10 mL of methanol was added to dissolve the solid, followed by the addition of few drops of concentrated sulfuric acid. The mixture was heat to reflux at 70° C. for 12 hours, then cooled to room temperature. After the reaction was completed, the solvent was dried by rotary evaporation, and then the mixture was washed with dichloromethane and water. An organic layer was collected and dried with magnesium sulfate, followed by the purification by column chromatography to obtain a white solid. Yield: 1 g (98%). Positive EI-MS: ion clusters at m/z 516 [M]⁺.

To obtain NAP4-f, to a THF solution (40 ml) of NAP4-e (1.0 g, 2 mmol) in ice water bath, methylmagnesium bromide (CAS: 75-16-1) in THF (1.4 M, 20 ml) was added dropwise under nitrogen. The mixture was warmed to room temperature and stirred for 12 hours. Then, the mixture was evaporated to dryness via vacuum and purified by reverse phase column chromatography using water as eluent, followed by washing with DCM to give red solid. Yield: 0.786 g (78%). Positive EI-MS: ion clusters at m/z 516 [M]⁺.

To obtain NAP4-g, in a 100 mL round-bottomed flask equipped with a magnetic stirrer, NAP4-f (0.78 g, 1.5 mmol), p-toluenesulfonic acid monohydrate (0.285 g, 1.5 mmol) and THF (30 ml) of were added. The mixture was heated to reflux for 8 hours. Then, the mixture was evaporated to dryness via vacuum and purified by reverse phase column chromatography using water as eluent, followed by washing with DCM to give white solid. Yield: 0.515 g (69%). Positive EI-MS: ion clusters at m/z 498 [M]⁺.

To obtain the final product of NAP4W, In a 100 mL round-bottomed flask equipped with a magnetic stirrer, NAP4-g (0.5 g, 1 mmol), potassium carbonate (1.38 g, 10 mmol), sodium 3-bromopropanesulfonate (CAS: 55788-44-8) (2.25 g, 10 mmol) and THF (50 mL) were added. The mixture was heated to reflux for 24 hours. Then, the mixture was acidified by addition of HCl until the pH reach 5. Then, the solvent was removed to dryness via vacuum and purified by reverse phase column chromatography using water as eluent, followed by washing with DCM to give pale yellow product. Yield: 0.695 g (80%). Negative ESI-MS: ion clusters at m/z 287 [M-3H]³⁻, 431 [M-2H]²⁻, 863 [M-H]⁻.

### Synthesis of NAP5W

A schematic diagram of the synthesis of NAP5W is as below:

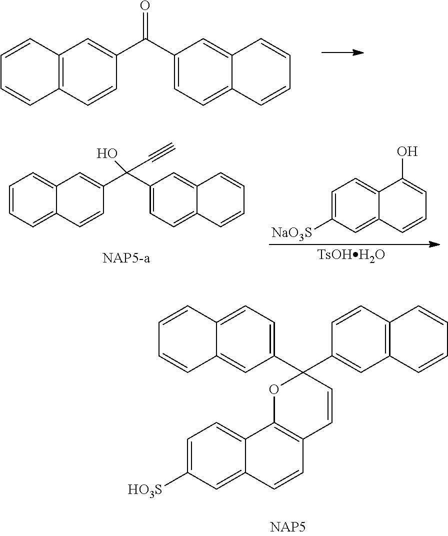

To obtain NAP5-a, to a THF solution (150 ml) of 2,2′-dinaphthyl ketone (CAS: 613-56-9) (20 g, 70.8 mmol) in ice water bath, sodium acetylide suspension (18 wt %) in THF solution was added dropwise under nitrogen. The mixture was warmed to room temperature and stirred for 6 hours. Then, the mixture was evaporated to dryness via vacuum and purified by reverse phase column chromatography using water as eluent, followed by washing with DCM to give pale yellow oil. Yield: 10 g (46%). Positive EI-MS: ion clusters at m/z 308 [M]⁺.

To obtain the final product of NAP5W, in a 250 mL round-bottomed flask equipped with a magnetic stirrer, NAP5-a (10 g, 32.4 mmol), sodium 6-hydroxynaphthalene-2-sulfonate (8 g, 32.4 mmol), p-toluenesulfonic acid monohydrate (0.04 g, 0.2 mmol) and THF (100 ml) were added. The mixture was heated to reflux for 2 hours. Then, the mixture was evaporated to dryness via vacuum and purified by reverse phase column chromatography using water as eluent, followed by washing with DCM to give pale yellow product. Yield: 9.86 g (59%). Negative ESI-MS: ion clusters at m/z 513 [M-H]⁻.

### Synthesis of NAP6W

A schematic diagram of the synthesis of NAP6W is as below:

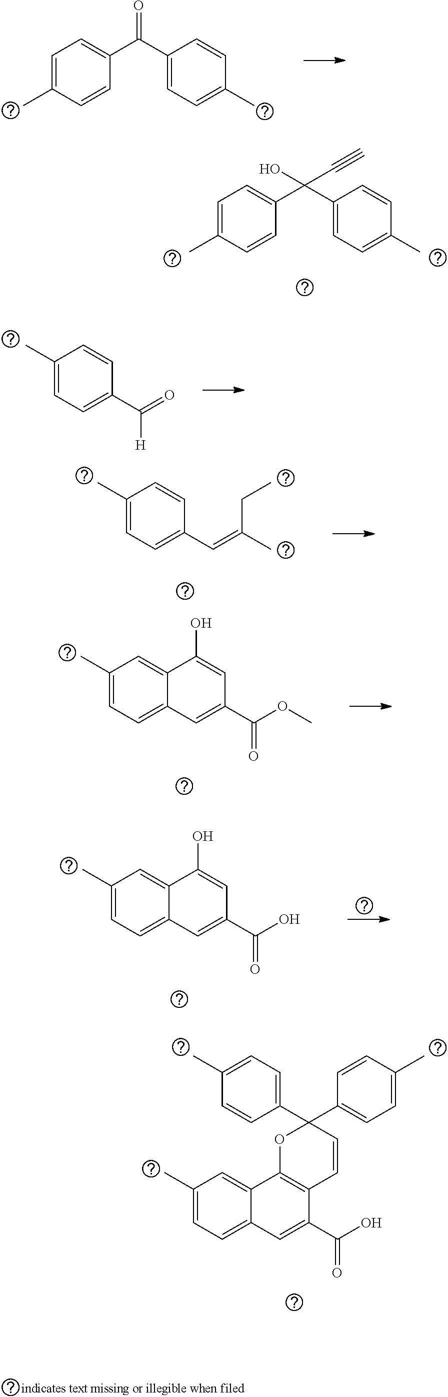

To obtain NAP6-a, to a THF solution (150 ml) of 4,4′-Diaminobenzophenone (CAS: 611-98-3) (20 g, 94.2 mmol) in ice water bath, sodium acetylide suspension (18 wt %) in THF solution was added dropwise under nitrogen. The mixture was warmed to room temperature and stirred for 6 hours. Then, the mixture was evaporated to dryness via vacuum and purified by reverse phase column chromatography using water as eluent, followed by washing with DCM to give pale yellow oil. Yield: 103.9 g (62%). Positive ESI-MS: ion clusters at m/z 120 [M+2H]²⁺, 239 [M+H]⁺.

To obtain NAP6-b, in a 500 mL round-bottomed flask equipped with a magnetic stirrer in ice water bath, sodium 4-formylbenzenesulfonate (CAS: 13736-22-6) (50 g, 240 mmol) and diglyme (60 ml) were added. Then, dimethyl succinate (CAS: 106-65-0) (45.6 g, 312 mmol) and potassium tert-butoxide (30.5 g, 271 mmol) (separated in 3 portions) were added. The mixture was stirred at room temperature for 4 hours. Then, the mixture was acidified by addition of HCl until the pH reach 5. Then, the mixture was evaporated to dryness via vacuum and purified by reverse phase column chromatography using water as eluent, followed by washing with DCM to give brown solid. Yield: 48.3 g (67%). Negative ESI-MS: ion clusters at m/z 299 [M-H]⁻.

To obtain NAP6-c, in a 250 mL round-bottomed flask equipped with a magnetic stirrer, NAP6-b (40 g, 133 mmol) was mixed with acetic anhydride (100 mL) and heated to reflux for 3 hours. Then, the mixture was evaporated to dryness via vacuum and purified by reverse phase column chromatography using water as eluent, followed by washing with DCM to give beige solid. Yield: 14.7 g (39%). Negative ESI-MS: ion clusters at m/z 281 [M-H]⁻.

To obtain NAP6-d, In a 250 mL round-bottomed flask equipped with a magnetic stirrer, NAP6-c (10 g, 35.4 mmol) was dissolved in THF (100 mL), followed by the addition of sodium hydroxide (14 g, 354 mmol) in water (30 mL). The mixture was warmed to 45° C. for 24 hours. Then, the mixture was acidified by addition of HCl until the pH reach 5. And the mixture was evaporated to dryness via vacuum and purified by reverse phase column chromatography using water as eluent, followed by washing with DCM to give white solid. Yield: 9 g (95%). Negative ESI-MS: ion clusters at m/z 133 [M-2H]²⁻ and 267 [M-H]⁻.

To obtain the final product of NAP6W, in a 250 mL round-bottomed flask equipped with a magnetic stirrer, NAP6-d (9 g, 33.5 mmol), NAP6-a (8 g, 33.5 mmol), p-toluenesulfonic acid monohydrate (6.37 g, 33.5 mmol) and THF (100 ml) were added. The mixture was heated to reflux for 12 hours. Then, the mixture was evaporated to dryness via vacuum and purified by reverse phase column chromatography using water as eluent, followed by washing with DCM to give pale yellow product. Yield: 8.35 g (51%). Positive ESI-MS: ion clusters at m/z 489 [M+H]⁺. Negative ESI-MS: ion clusters at m/z 487 [M-H]⁻.

### Synthesis of NAP7W

A schematic diagram of the synthesis of NAP7W is as below:

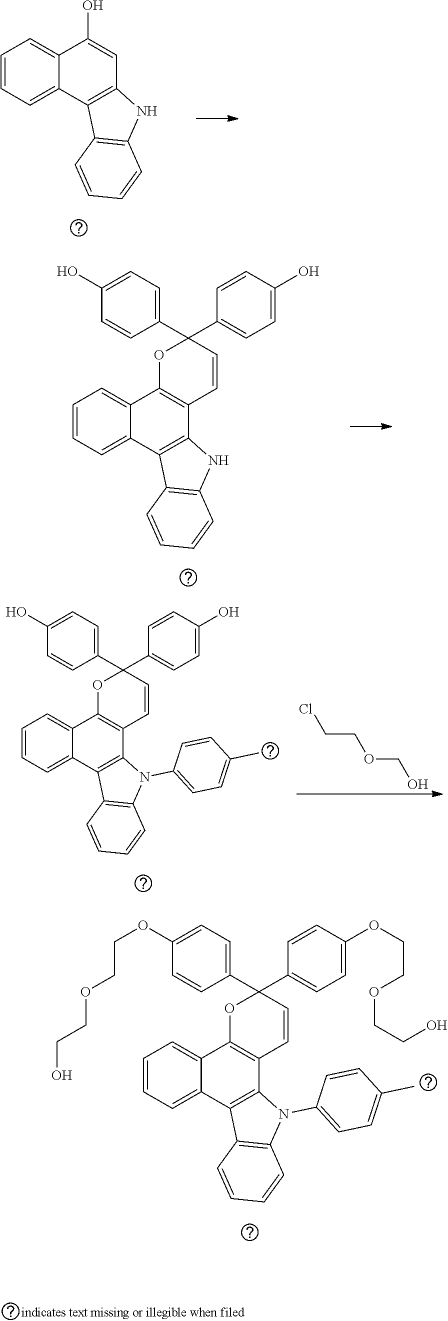 [SMILES: Oc1cc2[nH]c3ccccc3c2c2ccccc12 ; Oc1ccc(C2(c3ccc(O)cc3)C=Cc3c(c4ccccc4c4c3[nH]c3ccccc34)O2)cc1 ; Cc1ccc(-n2c3ccccc3c3c4ccccc4c4c(c32)C=CC(c2ccc(O)cc2)(c2ccc(O)cc2)O4)cc1 ; OCOCCCl ; Cc1ccc(-n2c3ccccc3c3c4ccccc4c4c(c32)C=CC(c2ccc(OCCOCCO)cc2)(c2ccc(OCCOCCO)cc2)O4)cc1]

To obtain NAP7-a, in a 500 mL round-bottomed flask equipped with a magnetic stirrer, NAP3-d (20 g, 85.8 mmol), 4,4′-dihydroxybenzophenone (18.36 g, 85.8 mmol), p-toluenesulfonic acid monohydrate (16.3 g, 33.5 mmol) and THF (250 ml) were added. The mixture was heated to reflux for 12 hours. Then, the solvent then was removed to dryness via vacuum, followed by extraction with DCM and water. The organic layer was collected and was dried by magnesium sulfate. Then, the crude product was purified by column chromatography using DCM/MeOH (10:1) as eluent to give pale yellow product. Yield: 13.68 g (35%). Negative ESI-MS: ion clusters at m/z 226 [M-2H]²⁻ and 454 [M-H]⁻.

To obtain NAP7-b, in a 250 mL round-bottomed flask equipped with a magnetic stirrer, NAP7-a (10 g, 22 mmol) was added and dissolve in THF (100 mL). And then THF suspension (50 mL) of sodium hydride (2.64 g, 110 mmol) was slowly added at 0° C., followed by stirring at 0° C. for 10 minutes. 4-Fluorobenzenesulfonic acid (CAS: 368-88-7) (4.65 g, 26.4 mmol) was then slowly added into the reaction mixture. After the addition, the reaction was allowed to stir at room temperature for 4 hours. After the reaction completed, ethanol was added to quench the reaction. The solvent was removed by vacuum. The mixture was then purified by reverse phase column chromatography using water as eluent and the white solid was obtained. Yield: 8.7 g (65%). Negative ESI-MS: ion clusters at m/z 610 [M-H]⁻.

To obtain the final product of NAP7W, in a 100 mL round-bottomed flask equipped with a magnetic stirrer, NAP7-b (5 g, 8.18 mmol), potassium carbonate (11.3 g, 81.8 mmol), 2-(2-chloroethoxy)ethanol (2.13 g, 17.2 mmol) and ACN (50 mL) were added. The mixture was heated to reflux for 24 hours. Then, the solvent was removed to dryness via vacuum and purified by reverse phase column chromatography using water as eluent, followed by washing with DCM to give pale yellow product. Yield: 4.7 g (73%). Negative ESI-MS: ion clusters at m/z 787 [M-H]⁻.

### Synthesis of NAP8W

A schematic diagram of the synthesis of NAP8W is as below:

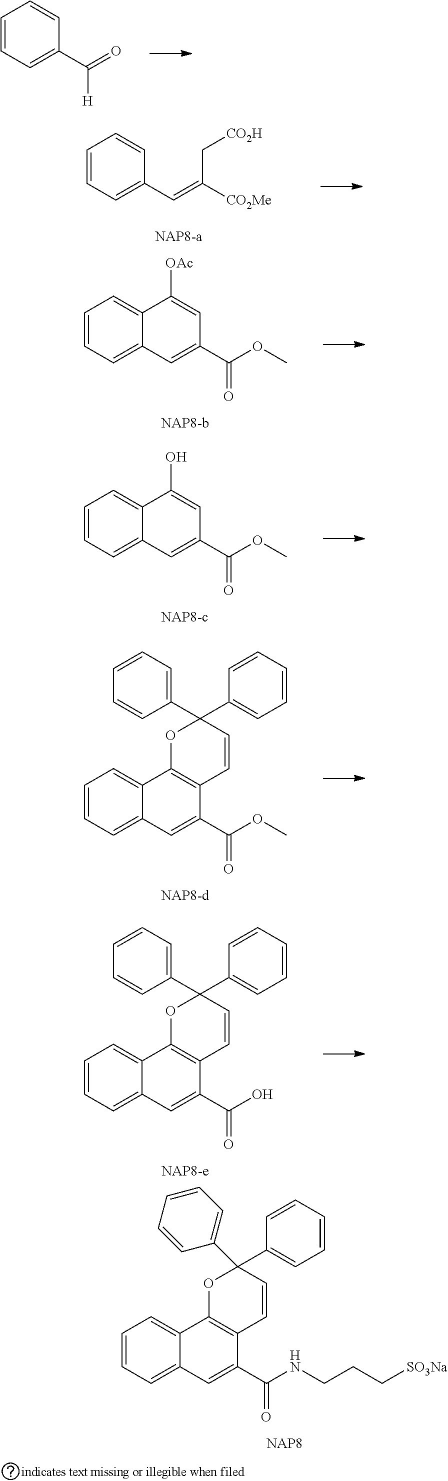

To obtain NAP8-a, to a THF solution (50 ml) of benzaldehyde (CAS: 100-52-7) (4 mL, 20 mmol) in water bath, dimethyl succinate (5.2 ml, 20 mmol) and potassium tert-butoxide (4.6 g, 20 mmol) (separated in 3 portions) were added. The mixture was stirred at room temperature for 3 hours. The mixture was extracted with ethyl acetate/H₂O, where the aqueous layer was acidified by addition of HCl (1 M aq. 28 ml) and the organic layer was dried over MgSO₄ and evaporated to dryness and yellow oil was obtained. Yield: 5.5 g (64%). Positive EI-MS: ion clusters at m/z 221 [M+H]⁺.

To obtain NAP8-b, in a 100 mL round-bottomed flask equipped with a magnetic stirrer, NAP8-a (5.5 g, 24.4 mmol) was mixed with sodium acetate (2.0 g, 24.4 mmol) and acetic anhydride (16.8 ml) and heated to reflux for 3 hours. The mixture was extracted with ethyl acetate/H₂O and washed with NaOH (0.5 M aq.) to neutral. The organic layer was dried over MgSO₄ and evaporated to dryness. The solid residue was purified by column chromatography in SiO₂ using n-hexane/ethyl acetate (8.5:1) as eluent to give beige solid. Yield: 2.9 g (48%). Positive EI-MS: ion clusters at m/z 245 [M+H]⁺.

To obtain NAP8-c, in a 100 mL round-bottomed flask equipped with a magnetic stirrer, NAP8-b (2.9 g, 11.6 mmol) was dissolved in ethanol (40 ml) and potassium carbonate (1.6 g, 11.6 mmol) was added. The mixture was stirred at room temperature for 3 hours. It was then filtered and evaporated to dryness. The solid residue was purified by column chromatography in SiO₂ using n-hexane/ethyl acetate (4:1) as eluent to give white solid. Yield: 1.7 g (70%). Positive EI-MS: ion clusters at m/z 203 [M+H]⁺.

To obtain NAP8-d, to a toluene solution (30 ml) of NAP8-c (1.68 g, 8.3 mmol), 1,1-diphenyl-2-propyn-1-ol (1.73 g, 8.3 mmol) and p-toluenesulfonic acid monohydrate (0.08 g, mmol) were added. The mixture was heated to reflux for 6 hours. The mixture was evaporated to dryness and purified by column chromatography in SiO₂ using n-hexane/ethyl acetate (9:1) as eluent, followed by washing with ethanol to give pale yellow solid. Yield: 1 g (31%). Positive EI-MS: ion clusters at m/z 393 [M+H]⁺.

To obtain NAP8-e, in a 250 mL round-bottomed flask equipped with a magnetic stirrer, NAP8-d (1 g, 2.55 mmol) was dissolved in THF (30 mL), followed by the addition of sodium hydroxide (1.02 g, 25.5 mmol) in water (10 mL). The mixture was warmed to 45° C. for 24 hours. Then, the mixture was acidified by addition of HCl until the pH reach 5. And the mixture was evaporated to dryness via vacuum and purified by reverse phase column chromatography using ACN as eluent, followed by washing with DCM to give white solid. Yield: 0.9 g (93%). Positive EI-MS: ion clusters at m/z 379 [M+H]⁺.

To obtain the final product of NAP8W, in a 100 mL round-bottomed flask equipped with a magnetic stirrer, 1-(3-Dimethylaminopropyl)-3-ethylcarbodiimide hydrochloride (EDC.HCl CAS: 25952-53-8) (2 g, 104 mmol) and 4-dimethylaminopyridine (DMAP, CAS: 1122-58-3) (0.382 g, 31.3 mmol) were mixed into a 1000-mL round bottom flask and mix solvent THF:H2O (250:50 v/v) (10 mL) was added to dissolve the solid. NAP1-COOH (3.95 g, 104 mmol) and 3-aminopropane-1-sulfonic acid (CAS: 3687-18-1) (1.6 g, 115 mmol) were then added to the EDC.HCl and DMAP. The reaction was stirred and was warmed up to 50° C. for 24 hours. Then, 1 M NaOH (aq) (20 mL) was added to the reaction mixture. The solvent THF was removed by rotary evaporation. Deionized water (20 mL) was then added to the mixture. The mixture was then centrifuged to remove the small white power. Then, water was removed by evaporation to obtain the red solid. Yield: 18.2 g (35%).

### 3. UV Absorption Spectra of Ring Closed Form and Ring Open Form of the Selected Photochromic Naphthopyrans

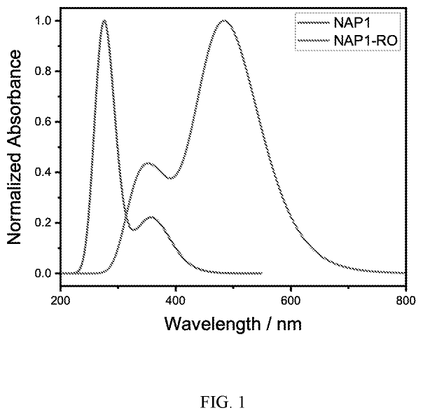

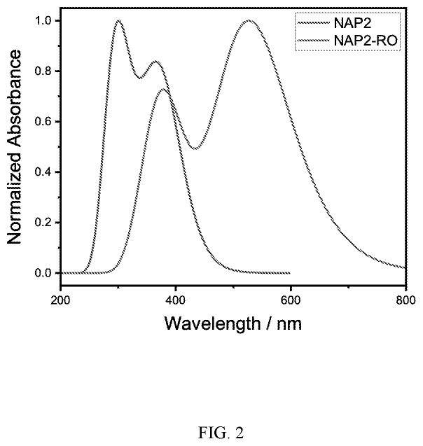

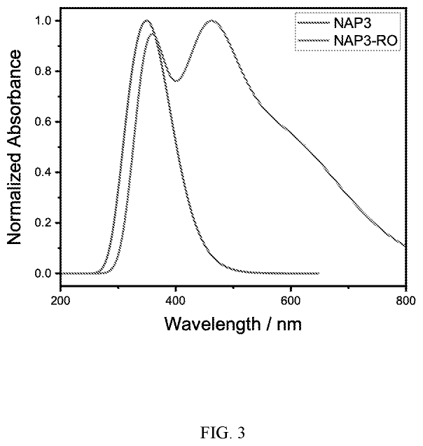

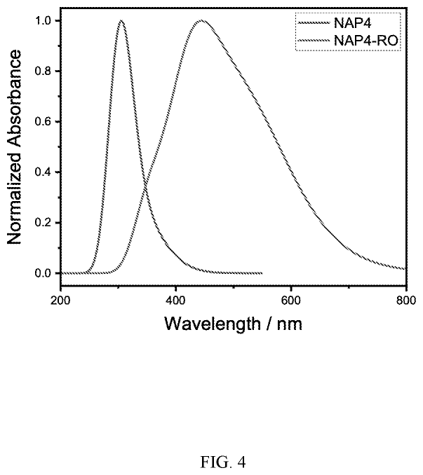

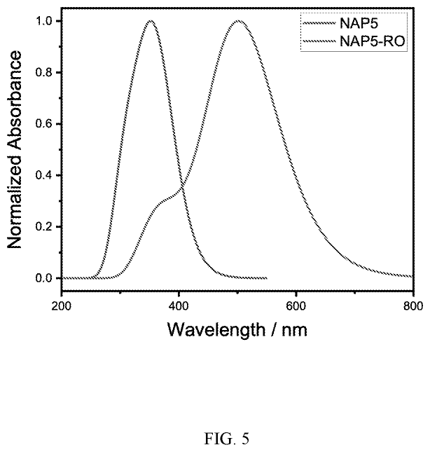

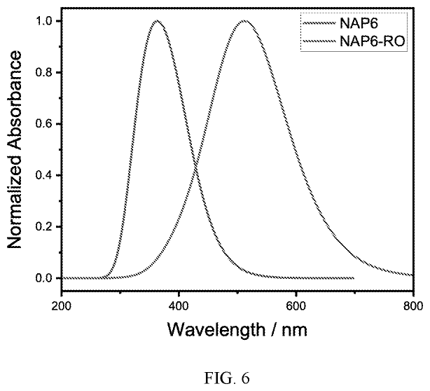

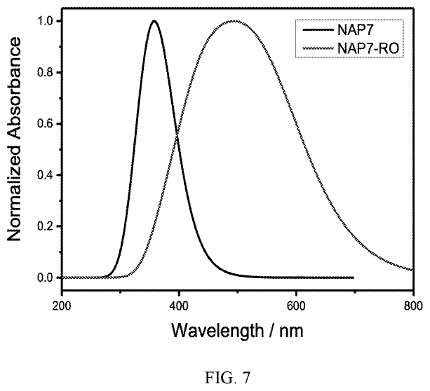

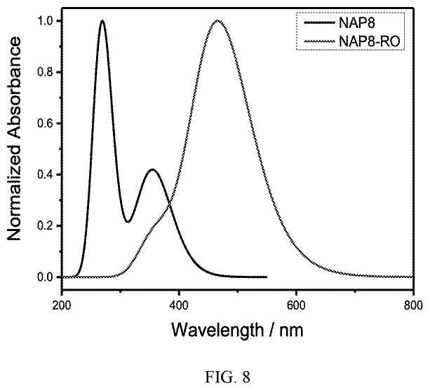

The stimulated UV/visible light absorption spectra of the ring-closed and ring-open form pairs of the selected photochromic naphthopyrans were stimulated by Gaussian 16W. FIGS. 1-8 corresponds to the UV absorption spectra of NAP1W/NAP1-RO, NAP2W/NAP2-RO, NAP3W/NAP3-RO, NAP4W/NAP4-RO, NAP5W/NAP5-RO, NAP6W/NAP6-RO, NAP7W/NAP7-RO and NAP8W/NAP8-RO pairs respectively.

It should be noted that the UV absorption spectra only corresponds to the first six lowest singlet states.

It should be apparent to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “includes,” “including,” “comprises,” and “comprising” should be interpreted as referring to elements, components, or steps in a non-exclusive manner, indicating that the referenced elements, components, or steps may be present, or utilized, or combined with other elements, components, or steps that are not expressly referenced.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

1. A water-soluble photochromic naphthopyran compound having a general structural formula represented by Formula (1): 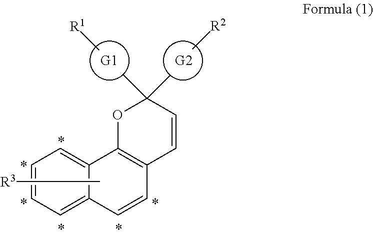
wherein G1 is selected from the group consisting of an aromatic containing 5 to 20 ring atoms, a heteroaromatic containing 5 to 20 ring atoms, or a non-aromatic ring system containing 5 to 20 ring atoms; G1 has a substituent R¹and the R¹ is the same or different in multiple occurrences;
wherein G2 is selected from the group consisting of an aromatic containing 5 to 20 ring atoms, a heteroaromatic containing 5 to 20 ring atoms, or a non-aromatic ring system containing 5 to 20 ring atoms; G2 has a substituent R², and the R² is the same or different in multiple occurrences;
wherein “*” represent possible bonding locations of Formula (1) with following chemical formula: 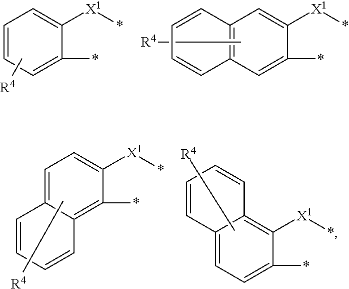
wherein X¹ is selected from CR⁵CR⁶, O, S, NR⁷, Se, S(═O)₂, C═O, S═O, PR⁸;
wherein R¹, R², R³, R⁴, R⁵, R⁶, R⁷, R⁸ are independently selected from the group consisting of hydrogen, deuterium, a halogen atom, water-solubilizing group, —OCH₃, —NO₂, —C≡N, —N≡C, a linear alkyl group containing 1 to 20 carbon atoms, a branched alkyl group containing 1 to 20 carbon atoms, and a linear alkenyl group containing 1 to 20 carbon atoms, a branched alkenyl group containing 1 to 20 carbon atoms, an alkane ether group containing 1 to 20 carbon atoms, an aromatic containing 1 to 20 carbon atoms, a heteroaromatic containing 1 to 20 carbon atoms or a non-aromatic ring system containing 1 to 20 carbon atoms; and
wherein R¹, R², R³, R⁴, R⁵, R⁶, R⁷, R⁸ independently contain at least one water-solubilizing group selected from —SO₃H, —COOH, —OH, —NH₂, —NCₙH₂ₙ₊₁H⁺, —N(CₙH₂ₙ₊₁)₃⁺, —SO₃Na, —OSO₃H, —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin.

2. The photochromic naphthopyran compound according to claim 1, wherein G1 and G2 in Formula (1) is selected from the following formulae: 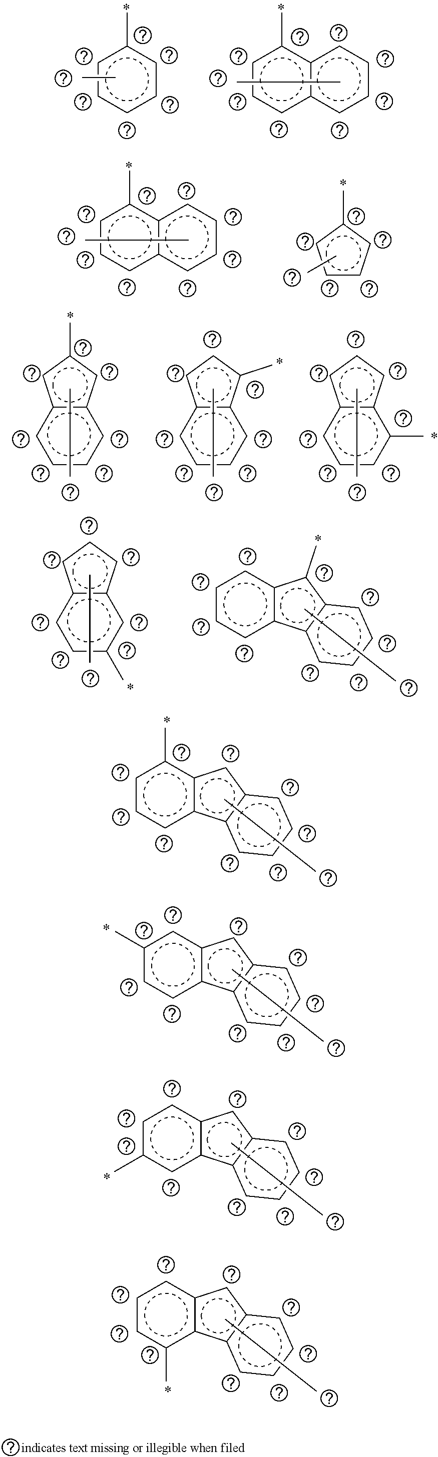
wherein # represents the bonding location in Formula (1);
wherein Z¹-Z⁹ are independently or jointly selected from the group consisting of carbon, nitrogen, oxygen, silicon, boron, sulfur or phosphorus atom;
wherein R⁹ are independently or jointly selected from the group consisting of hydrogen, deuterium, a halogen atom, —OCH₃, —NO₂,—C≡N, —N≡C, —NPh₂, N-carbazole, dibenzofuran, dibenzothiophene, —SO₃H, —OH, —NCₙH₂ₙ₊₁H⁺, —N(CₙH₂ₙ₊₁)₃⁺, —SO₃Na, —OSO₃H , —SO₂NH₂, —PO₃H₂, —PO₃Na₂, —SH, —SeH, —CONH₂, —(OCₙH₂ₙ₊₁)ₘOH, —OCₙH₂ₙSO₃H, —NH(CₙH₂ₙ₊₁)SO₃H, —O-morpholinylethyl, —O-glucoside, —O-crown ether, —O-cyclodextrin or a linear alkyl group containing 1 to 20 carbon atoms, a branched alkyl group containing 1 to 20 carbon atoms, and a linear alkenyl group containing 1 to 20 carbon atoms, a branched alkenyl group containing 1 to 20 carbon atoms, an alkane ether group containing 1 to 20 carbon atoms, an aromatic containing 1 to 20 carbon atoms, a heteroaromatic containing 1 to 20 carbon atoms or a non-aromatic ring system containing 1 to 20 carbon atoms.

3. The photochromic naphthopyran compound according to claim 1, containing a structure represented by one of general formulas (A-1) to (A-15): 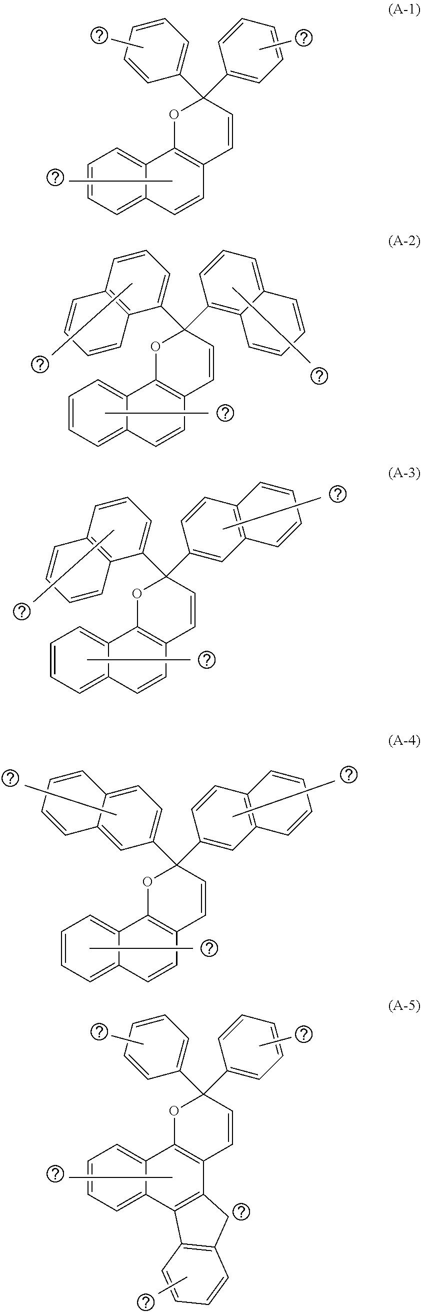 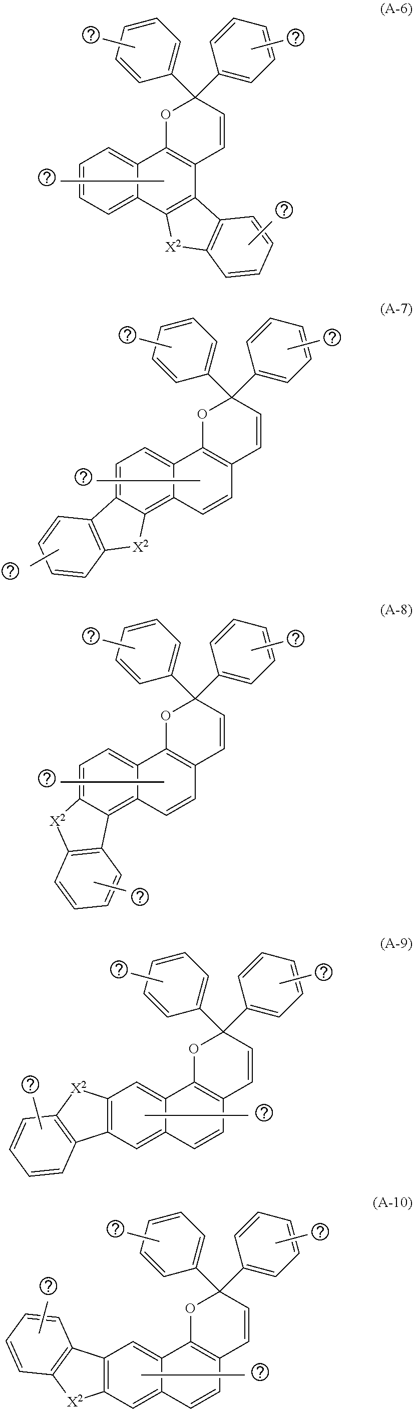 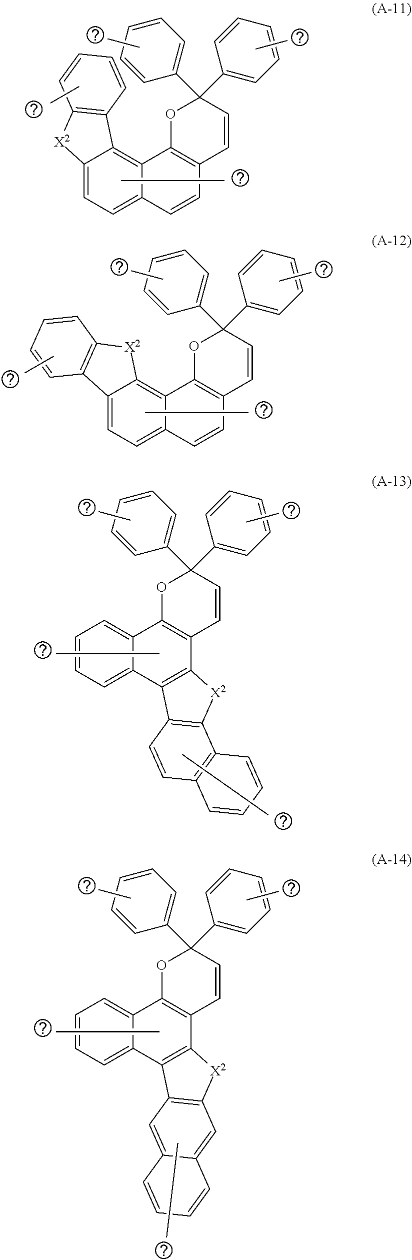 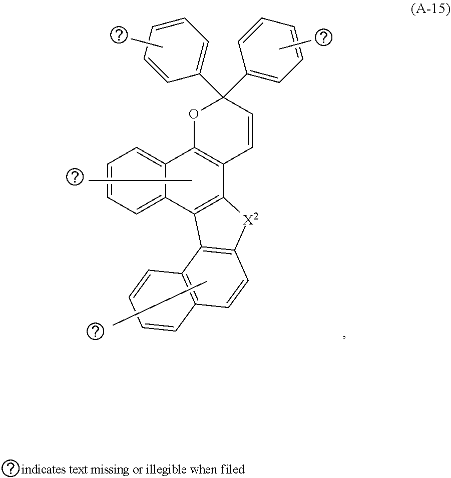
wherein R¹⁰, R¹¹, R¹², R¹³ are independently or jointly selected from the group consisting of hydrogen, deuterium, a halogen atom, —OCH₃, —NO₂, —C≡N, —N≡C, —NPh₂, N-carbazole, dibenzofuran, dibenzothiophene, —SO₃H, —OH, —NCₙH₂ₙ₊₁H⁺, —N(CₙH₂ₙ₊₁)₃⁺, —SO₃Na, —OSO₃H , —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin or a linear alkyl group containing 1 to 20 carbon atoms, a branched alkyl group containing 1 to 20 carbon atoms, and a linear alkenyl group containing 1 to 20 carbon atoms, a branched alkenyl group containing 1 to 20 carbon atoms, an alkane ether group containing 1 to 20 carbon atoms, an aromatic containing 1 to 20 carbon atoms, a heteroaromatic containing 1 to 20 carbon atoms or a non-aromatic ring system containing 1 to 20 carbon atoms;
wherein X² is selected from CR⁵CR⁶, O, S, NR⁷, Se, S(═O)₂, C═O, S═O, PR⁸.

4. The photochromic naphthopyran compound according to claim 3, at least one R¹⁰ or R¹¹ or R¹² or R¹³ is selected from —SO₃H, —OH, —NCₙH₂ₙ₊₁H⁺, —N(CₙH₂ₙ₊₁)₃⁺, —SO₃Na, —OSO₃H, —SO₂NH₂, —PO₃H₂, —PO₃Na₂, —SH, —SeH, —CONH₂, —(OCₙH₂ₙ)ₘOH, —OCₙH₂ₙSO₃H, —NH(CₙH₂ₙ₊₁)SO₃H, —O-morpholinylethyl, —O-glucoside, —O-crown ether, —O-cyclodextrin.

5. The photochromic naphthopyran compound according to claim 1, wherein the naphthopyran compound is selected from the following chemical structures:

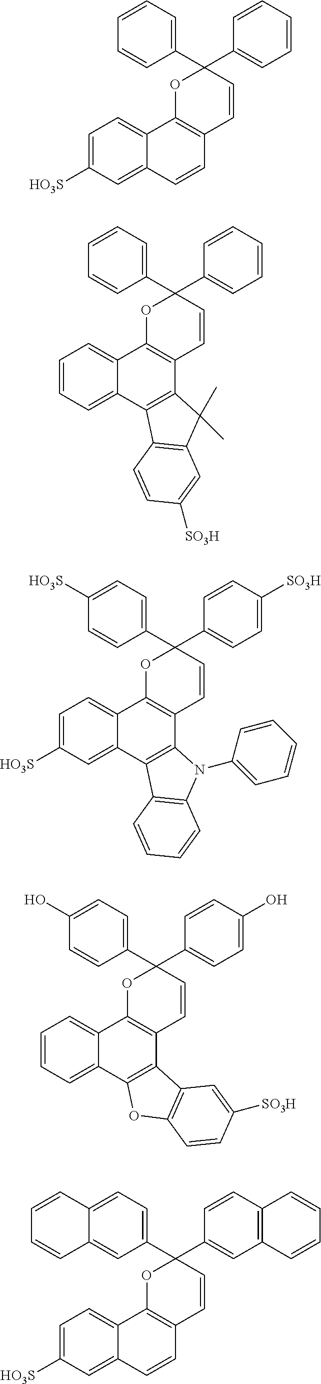

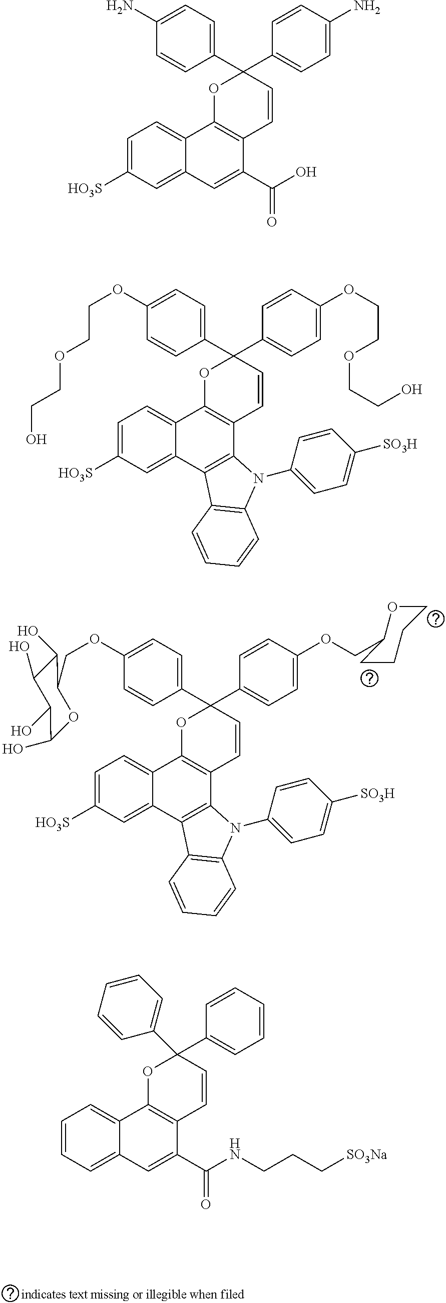

6. The photochromic naphthopyran compound according to claim 1, wherein the conformation of the molecule can be changed to ring-open form upon UV or visible light irradiation, and can be thermodynamically changed backward to the ring-close form:

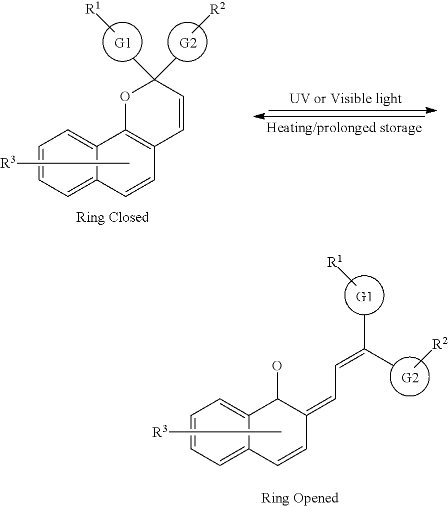

7. The photochromic naphthopyran compound according to claim 1, wherein the water solubility of the compound is greater than or equal to 10 mg L⁻¹.

8. A photochromic mixture comprising at least one photochromic naphthopyran compound according to claim 1, wherein the weight percent of the photochromic naphthopyran compound in the mixture is greater than 0.01%.

9. The photochromic mixture according to claim 8, comprising the water solubility greater than or equal to 10 mg L⁻¹.

10. The photochromic mixture according to claim 8, wherein the mixture displays photochromic property upon UV or visible light irradiation, and wherein the photochromism is thermodynamically reversible.

11. The photochromic naphthopyran compound according to claim 1, wherein an absorption peak in UV/v is absorption spectrum ranges from 100 nm to 1500 nm.
